(19) United States
(12) Patent Application Publication
(10) Pub. No.: US 2025/0099024 A1
(43) Pub. Date: Mar. 27, 2025

(54) Spectral image-based melanoma determination method, detection method, and device supporting same

(71) Applicant: SK Planet Co., Ltd., Seongnam-si (KR)

(72) Inventor: Yeonghyeon PARK, Cheonan-si (KR)

(21) Appl. No.: 18/970,931

(22) Filed: Dec. 6, 2024

Related U.S. Application Data
(63) Continuation of application No. PCT/KR2023/019422, filed on Nov. 29, 2023.

(30) Foreign Application Priority Data
Dec. 6, 2022 (KR) 10-2022-0169196
Dec. 6, 2022 (KR) 10-2022-0169197

(51) Int. Cl. A61B 5/00; G06T 7/00; G06T 7/11; G06T 7/194; H04N 23/60

(52) U.S. Cl. CPC A61B 5/444; A61B 5/0075; G06T 7/0014; G06T 7/11; G06T 7/194; H04N 23/64; G06T 2207/20021; G06T 2207/30088; G06T 2207/30096

(57) ABSTRACT

The present invention relates to a spectral image-based melanoma determination method, a detection method, and a device supporting same. The present invention may provide a spectral image-based melanoma determination method and a device for operating same, the method comprising: a step for controlling a spectral camera and acquiring a current spectral image of an examination subject in which at least one skin lesion has appeared; a step in which a processor separates a foreground area in which the at least one skin lesion has appeared and a background area other than the foreground area in the current spectral image; and a step in which the processor compares a pre-stored reference model with at least a portion of the separated foreground area and background area and outputs a melanoma determination result of the examination subject.

### CROSS-REFERENCE TO RELATED APPLICATIONS

This application is a bypass-continuation of International PCT Application No. PCT/KR2023/019422, filed on Nov. 29, 2023, which claims priority to Republic of Korea Patent Application No. 10-2022-0169196 filed on Dec. 6, 2022, and Republic of Korea Patent Application No. 10-2022-0169197, filed on Dec. 6, 2022, which are incorporated by reference herein in their entirety.

### TECHNICAL FIELD

The present invention relates to melanoma discrimination or examination, and more particularly, to a melanoma discrimination or examination technique using a non-destructive spectral image.

### BACKGROUND ART

Melanoma is defined as the most dangerous type of skin cancer among skin diseases. Melanoma can be cured in most cases if detected early, but if not detected early, it spreads to other areas, making it difficult to treat and causing a risk of death. There are self-diagnosis methods for melanoma, such as checking for changes in the pattern of spots on the skin and the shape of newly appeared spots, but it is not easy for the average person to distinguish between normal and abnormal spots. Recently, thanks to technological advancements and continuous research, various methods for diagnosing melanoma have been proposed, and the ABCD method is suggested as a clinical methodology. The ABCD method is a method in which medical professional directly observe the patient's lesion and identify the suspicious area by observing the asymmetry, border irregularity, color irregularity, and diameter of the spot or mole. Once the suspicious area is identified, the skin is incised and a tissue biopsy is performed to confirm whether it is melanoma.

As such, since the method of isolating skin tissue suspected of being melanoma from the skin and examining it is mainly used to diagnose the occurrence of melanoma, it takes a lot of time to determine whether melanoma is present, and the person being examined may feel uncomfortable with the incision of skin tissue.

The number of patients with melanoma lesions is steadily increasing, but the number of medical professional performing observation and tissue examination of lesions is not easily increasing. Therefore, there is a shortage of personnel to identify melanoma lesions, and there is a problem that it costs a lot of money and takes a lot of time if medical professional conduct periodic tests for melanoma.

### SUMMARY

The present invention is intended to provide a method and device for melanoma discrimination based on a spectral image, which can efficiently and precisely separate the foreground and background of the spectral image in melanoma discrimination to clearly distinguish the area for melanoma discrimination, thereby producing more accurate discrimination results while improving the amount of computation.

In addition, the present invention is intended to provide a method and device for melanoma examination based on a spectral image, which can quickly and accurately perform melanoma examination of a patient using the spectral image in melanoma examination, and which uses a non-destructive scheme to eliminate the process of skin incision, etc.

However, the objects of the present invention are not limited to the above objects, and other objects not mentioned can be clearly understood from the description below.

To achieve the above objects, a melanoma discrimination device according to the present invention may include a spectral camera acquiring a spectral image of an examination target containing at least one skin lesion, and a processor functionally connected to the spectral camera. The processor may be configured to control the spectral camera to acquire a current spectral image of the examination target, to separate a foreground region where the at least one skin lesion has occurred and a background region other than the foreground region from the current spectral image, and to compare at least a part of the separated foreground and background regions with a pre-stored reference model to output a melanoma discrimination result for the examination target.

Specifically, the processor may be configured to detect an adjacency matrix based on a plurality of dimensional vectors and edges corresponding to distance values between the plurality of dimensional vectors by applying a nearest neighbor technique to the current spectral image, and to extract the foreground region based on edges greater than or equal to a predefined reference value in the adjacency matrix.

Specifically, the processor may be configured to extract a cluster whose similarity with a cluster corresponding to a spectrum of the foreground region is higher than or equal to a predefined reference value, from the reference model, and to output the melanoma discrimination result for the examination target, based on the cluster extracted from the reference model.

Specifically, the processor may be configured to detect directionality of the cluster corresponding to the spectrum of the foreground region, to extract a cluster having a similar directionality within a certain range from the detected directionality, from the reference model, and to output the melanoma discrimination result for the examination target, based on the cluster extracted from the reference model.

Specifically, the processor may be configured to output the adjacency matrix to a display after visualization.

Specifically, the processor may be configured to output the melanoma discrimination result through cross-validation between a melanoma discrimination result based on the foreground region and a melanoma discrimination result based on both the foreground region and the background region.

A melanoma discrimination method according to an embodiment of the present invention may include, by a processor of a melanoma discrimination device, controlling a spectral camera to acquire a current spectral image of an examination target containing at least one skin lesion; by the processor, separating a foreground region where the at least one skin lesion has occurred and a background region other than the foreground region from the current spectral image; and by the processor, comparing at least a part of the separated foreground and background regions with a pre-stored reference model to output a melanoma discrimination result for the examination target.

Specifically, separating the foreground and background regions may include detecting an adjacency matrix based on a plurality of dimensional vectors and edges corresponding to distance values between the plurality of dimensional vectors by applying a nearest neighbor technique to the current spectral image; and extracting the foreground region based on edges greater than or equal to a predefined reference value in the adjacency matrix.

Specifically, outputting the melanoma discrimination result may include one of extracting a cluster whose similarity with a cluster corresponding to a spectrum of the foreground region is higher than or equal to a predefined reference value, from the reference model, and outputting the melanoma discrimination result for the examination target, based on the cluster extracted from the reference model; or detecting directionality of the cluster corresponding to the spectrum of the foreground region, extracting a cluster having a similar directionality within a certain range from the detected directionality, from the reference model, and outputting the melanoma discrimination result for the examination target, based on the cluster extracted from the reference model.

Specifically, outputting the melanoma discrimination result may include outputting the melanoma discrimination result through cross-validation between a melanoma discrimination result based on the foreground region and a melanoma discrimination result based on both the foreground region and the background region.

A server device supporting melanoma discrimination based on a spectral image according to an embodiment of the present invention may include a server communication circuit establishing a communication channel with a melanoma discrimination device, and a server processor functionally connected to the server communication circuit. The server processor may be configured to receive a current spectral image of an examination target containing at least one skin lesion from the melanoma discrimination device, to separate a foreground region where the at least one skin lesion has occurred and a background region other than the foreground region from the current spectral image, to perform melanoma discrimination on the examination target by comparing at least a part of the separated foreground and background regions with a pre-stored reference model, and to transmit a melanoma discrimination result to the melanoma discrimination device.

Specifically, the server processor may be configured to perform the melanoma discrimination through cross-validation between a melanoma discrimination result based on the foreground region and a melanoma discrimination result based on both the foreground region and the background region.

A melanoma examination device according to the present invention may include a spectral camera acquiring a spectral image of an examination target containing at least one skin lesion, and a processor functionally connected to the spectral camera. The processor may be configured to control the spectral camera to acquire a current spectral image of the examination target, to detect a latent vector by applying the current spectral image to a pre-stored reference model, and to determine whether the examination target is melanoma based on directionality and form of a latent representation corresponding to the latent vector.

Specifically, the processor may be configured to control the spectral camera so that a shooting angle and distance of the spectral camera with respect to the examination target become a predefined shooting angle and distance.

Specifically, the processor may be configured to determine a type of melanoma for the examination target based on the directionality and form of the latent representation, and to output information on the determined type of melanoma.

Specifically, the processor may be configured to determine that a magnitude of the skin lesion in the current spectral image is stronger the farther away the lesion is expressed from a center of the latent representation.

Specifically, the processor may be configured to, when the current spectral image contains a plurality of skin lesions, divide the image into regions including the plurality of skin lesions, to separate each skin lesion and a background region of a predetermined size surrounding each skin lesion in the divided regions, and to perform sequential melanoma examinations on each of the separated skin lesions and background regions.

Specifically, the processor may be configured to extract a cluster for a spectrum of the current spectral image, to detect a cluster of latent representation most similar to a cluster corresponding to the current spectral image from the reference model, to determine whether the cluster corresponding to the current spectral image is melanoma and a type of melanoma based on the cluster detected from the reference model, and to output determined information.

A melanoma examination method based on a spectral image according to an embodiment of the present invention may include, by a processor of a melanoma examination device, controlling a spectral camera to acquire a current spectral image of an examination target containing a skin lesion; detecting a latent vector by applying the current spectral image to a pre-stored reference model; and determining at least one of whether the examination target is melanoma and a type of melanoma, based on directionality and form of a latent representation corresponding to the latent vector.

Specifically, the method may further include outputting information including the determined at least one of whether the examination target is melanoma and the type of melanoma.

Specifically, detecting the latent vector may include, when the current spectral image contains a plurality of skin lesions, dividing the image into regions including the plurality of skin lesions; separating each skin lesion and a background region of a predetermined size surrounding each skin lesion in the divided regions; and sequentially detecting the latent vector on each of the separated skin lesions and background regions.

Specifically, determining may include extracting a cluster for a spectrum of the current spectral image; detecting a cluster of latent representation most similar to a cluster corresponding to the current spectral image from the reference model; and determining whether the cluster corresponding to the current spectral image is melanoma and a type of melanoma based on the cluster detected from the reference model.

A server device supporting melanoma examination based on a spectral image according to an embodiment of the present invention may include a server communication circuit establishing a communication channel with a melanoma examination device, and a server processor functionally connected to the server communication circuit. The server processor may be configured to receive a current spectral image of an examination target containing a skin lesion from the melanoma examination device, to detect a latent vector by applying the current spectral image to a pre-stored reference model, to determine at least one of whether the examination target is melanoma and a type of melanoma, based on directionality and form of a latent representation corresponding to the latent vector, and to transmit determined information to the melanoma examination device.

Specifically, the server processor may be configured to extract a cluster for a spectrum of the current spectral image, to detect a cluster of latent representation most similar to a cluster corresponding to the current spectral image from the reference model, and to determine whether the cluster corresponding to the current spectral image is melanoma and a type of melanoma based on the cluster detected from the reference model.

According to the present invention, the method and device for melanoma discrimination based on a spectral image can more accurately discriminating melanoma by precisely and accurately separating the foreground corresponding to a skin lesion from the background in the spectral image.

In addition, the method and device for melanoma examination based on a spectral image according to the present invention can reduce the human, time, and cost consumption of a direct lesion examination method by medical professional, can provide examination results for lesions quickly and accurately, and can minimize the burden on patients through a non-destructive examination scheme without incision of skin tissue.

In addition, various effects other than the effects described above can be directly or implicitly disclosed in the detailed description according to embodiments of the present invention to be described later.

### BRIEF DESCRIPTION OF THE DRAWINGS

FIG. 1 is a diagram showing an example of a system environment supporting a melanoma discrimination function according to the first embodiment of the present invention.

FIG. 2 is a diagram showing an example of components of a melanoma discrimination device according to the first embodiment of the present invention.

FIG. 3 is a diagram showing an example of components of the processor in FIG. 2.

FIG. 4 is a diagram showing an example of a spectral image for an examination target according to the first embodiment of the present invention.

FIG. 5 is a diagram showing an example of separation between foreground and background in the spectral image in FIG. 4.

FIG. 6 is a diagram showing an example of components of a server device according to the first embodiment of the present invention.

FIG. 7 is a diagram showing an example of a method for operating a melanoma discrimination device in relation to a melanoma discrimination method according to the first embodiment of the present invention.

FIG. 8 is a diagram showing another example of a method for operating a melanoma discrimination device in relation to a melanoma discrimination function according to the first embodiment of the present invention.

FIG. 9 is a diagram showing an example of a system environment supporting a melanoma examination function according to the second embodiment of the present invention.

FIG. 10 is a diagram showing an example of components of a melanoma examination device according to the second embodiment of the present invention.

FIG. 11 is a diagram showing an example of components of the processor in FIG. 10.

FIG. 12 is a diagram showing an example of a latent vector representation for melanoma data according to the second embodiment of the present invention.

FIG. 13 is a diagram showing an example of a melanoma examination function for an examination target according to the second embodiment of the present invention.

FIG. 14 is a diagram showing an example of components of a server device according to the second embodiment of the present invention.

FIG. 15 is a diagram showing an example of a method for operating a melanoma examination device in relation to a melanoma examination method according to the second embodiment of the present invention.

FIG. 16 is a diagram showing another example of a method for operating a melanoma examination device in relation to a melanoma examination function according to the second embodiment of the present invention.

### DETAILED DESCRIPTION

Now, embodiments of the present invention will be described in detail with reference to the accompanying drawings.

However, in the following description and the accompanying drawings, well known functions and components may not be described nor illustrated in detail to avoid obscuring the subject matter of the present invention. In addition, identical components are indicated with the same reference numerals as much as possible throughout the drawings

The terms or words used in the following description and drawings should not be interpreted as limited to their usual or dictionary meanings and should be interpreted as meanings and concepts that conform to the technical idea of the present invention based on the principle that the inventor can appropriately define the concept of the terms to best describe his or her invention. Therefore, embodiments described herein are only the most preferred embodiments of the present invention and do not represent all of the technical ideas of the present invention. Thus, it should be understood that there may be various equivalents and modified examples that can replace the embodiments at the time of filing this application.

In addition, terms including ordinal numbers such as first, second, etc. are used to describe various elements only for the purpose of distinguishing one element from another, and are not used to limit such elements. For example, without departing from the scope of the present invention, a second element may be named a first element, and similarly, a first element may also be named a second element.

In addition, terms used herein are only for describing specific embodiments and do not limit the present disclosure. The singular expression includes the plural expression unless the context clearly indicates otherwise. Also, the terms such as “comprise” and “include” used herein are intended to specify the presence of features, numerals, steps, operations, elements, components, or combinations thereof, which are disclosed herein, and should not be construed to exclude in advance the possibility of the presence or addition of other features, numerals, steps, operations, elements, components, or combinations thereof.

In addition, the terms such as “unit” and “module” used herein refer to a unit that processes at least one function or operation and may be implemented with hardware, software, or a combination of hardware and software. In addition, the terms “a”, “an”, “one”, “the”, and similar terms may be used as both singular and plural meanings in the context of describing the present invention (especially in the context of the following claims) unless the context clearly indicates otherwise.

In addition to the terms mentioned above, specific terms used in the following description are provided to help understanding of the present invention, and the use of such specific terms may be changed to other forms without departing from the technical meaning of the present invention.

Also, embodiments within the scope of the present invention include computer-readable media having computer-executable instructions or data structures stored on computer-readable media. Such computer-readable media can be any available media that is accessible by a general purpose or special purpose computer system. By way of example, such computer-readable media may include, but not limited to, RAM, ROM, EPROM, CD-ROM or other optical disk storage, magnetic disk storage or other magnetic storage devices, or any other physical storage medium that can be used to store or deliver certain program codes formed of computer-executable instructions, computer-readable instructions or data structures and which can be accessed by a general purpose or special purpose computer system.

### First Embodiment

Hereinafter, in the present invention, a melanoma discrimination function, a melanoma discrimination system environment supporting the same, and the types and roles of each component included therein will be described.

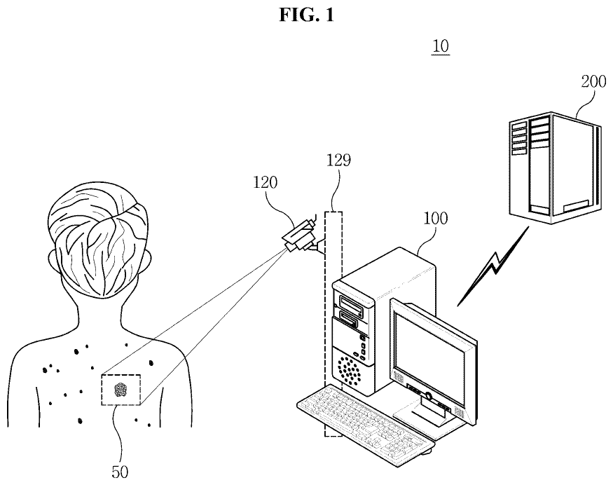

FIG. 1 is a diagram showing an example of a system environment supporting a melanoma discrimination function according to the first embodiment of the present invention.

Referring to FIG. 1, the system environment 10 that supports the melanoma discrimination function of the present invention may include an examination target 50 (e.g., at least a portion of the skin of a patient having a lesion (or skin lesion)), a melanoma discrimination device 100 capable of photographing at least partial area of the examination target 50, and a server device 200 capable of supporting the melanoma discrimination device 100. In the illustrated system environment 10, a communication channel is formed between the melanoma discrimination device 100 and the server device 200, and the server device 200 supports the melanoma discrimination device 100. This is, however, exemplary only and present invention is not limited thereto. In an alternative example, the system environment 10 may be configured based on one electronic device (e.g., the melanoma discrimination device 100) capable of executing an embedded program for performing spectral image collection and melanoma determination on the examination target 50 without using both the melanoma discrimination device 100 and the server device 200. In this case, the server device 200 may be omitted from the configuration of the system environment 10.

The examination target 50 may include at least a portion of the skin of a patient with at least one skin lesion. Although the patient's back is exemplarily shown as the examination target 50, the present invention is not limited thereto. For example, the examination target 50 may include any area where melanoma may occur. For example, the examination target 50 may be any skin area where melanoma may occur, such as the soles of the feet, palms of the hands, face, chest, abdomen, buttocks, genitals or surrounding areas, scalp, etc., and may also be at least a portion of an internal organ.

The melanoma discrimination device 100 can obtain a spectral image regarding the examination target 50. For example, the melanoma discrimination device 100 may be configured to approach the examination target 50 within a certain distance and capture a spectral image of the examination target 50 with a resolution of a certain level or higher. In this regard, the melanoma discrimination device 100 may include at least a spectral camera 120 and a mounting structure 129 that mounts the spectral camera 120 and enables at least one of linear motion and rotational motion of the spectral camera 120. The spectral camera 120 can capture a spectral image regarding the examination target 50 located at a certain angle and within a certain distance. The mounting structure 129 is configured to at least temporarily mount the spectral camera 120 and allow linear movement in at least one of the forward-backward, left-right, and up-down directions of the spectral camera 120 and/or rotational movement at a specific angle. Therefore, the melanoma discrimination device 100 can adjust the shooting range for the examination target 50 as needed. The melanoma discrimination device 100 can capture at least a region of the examination target 50 including at least one lesion to acquire a spectral image corresponding to the region. In addition, the melanoma discrimination device 100 can separate the foreground (e.g., the region of interest as a skin lesion region) and background (e.g., the skin region without a lesion other than the region of interest) of the acquired spectral image for the examination target 50, compare the separated data with a pre-stored reference model, and then perform melanoma discrimination based on the comparison result.

Alternatively, the melanoma discrimination device 100 may obtain a melanoma discrimination result for the examination target 50 through the server device 200. In this case, the melanoma discrimination device 100 does not perform foreground and background separation and model comparison analysis based on separated data for the spectral image. Instead, the melanoma discrimination device 100 may be configured to transmit the acquired spectral image to the server device 200, receive the melanoma discrimination result from the server device 200, and output the received result. In addition, when the melanoma discrimination device 100 is configured to independently perform melanoma discrimination and classification and result output without the server device 200, the melanoma discrimination device 100 may store in advance the reference model received from an external server device. The reference model may be a model generated by performing machine learning or unsupervised learning on various melanoma spectral images. The reference model may be generated based on various spectral images. The reference model may be generated based on data in which the foreground and background of various spectral images are separated, and may include at least one of a foreground reference model generated based only on the foregrounds, and an integrated reference model based on the separated foregrounds and backgrounds. For example, when the spectral images acquired through the spectral camera 120 and the examination records for the corresponding examination target 50 are accumulatively stored in a predefined amount or more, the melanoma discrimination device 100 may separate the foreground and background of the spectral images and directly generate a reference model based on unsupervised learning on at least a part of the separated data. Here, for the purpose of protecting information on personal examination records, the information used to generate the reference model may include only spectral images for the examination target 50 and examination results for the spectral images. Additionally or alternatively, the information used to generate the reference model may include only information other than information that can identify an individual, such as the age, gender, and weight of the examination target 50.

The server device 200 can establish a communication channel with the melanoma discrimination device 100. The server device 200 can receive at least one spectral image of the examination target 50 from the melanoma discrimination device 100 and perform melanoma discrimination on the received at least one spectral image. In this process, the server device 200 may pre-store a reference model for comparative analysis of the currently acquired spectral image. The reference model may be generated by separating the foreground and background from the spectral images collected and provided by the melanoma discrimination device 100 and performing unsupervised learning on the separated data, or may be received from a separate external server device that provides the reference model. The server device 200 can provide the result of comparative analysis with the reference model to the melanoma discrimination device 100. Meanwhile, if the resolution of the acquired spectral image is insufficient or a spectral image with a higher resolution is required, the server device 200 may request the melanoma discrimination device 100 to adjust the resolution for the examination target 50 with the corresponding lesion, and obtain a spectral image with improved resolution. In response, the melanoma discrimination device 100 may adjust the shooting angle and shooting distance of the spectral camera 120 to capture a new spectral image for the examination target 50 and provide it to the server device 200.

As described above, in the system environment 10 that supports the melanoma discrimination function according to the first embodiment of the present invention, the melanoma discrimination device 100 can acquire a spectral image of the examination target 50, separate the foreground and background of the spectral image, and perform a comparison with a pre-learned reference model based on the separated data, thereby obtaining the melanoma discrimination result of the examination target 50 more accurately and quickly through more accurate information comparison.

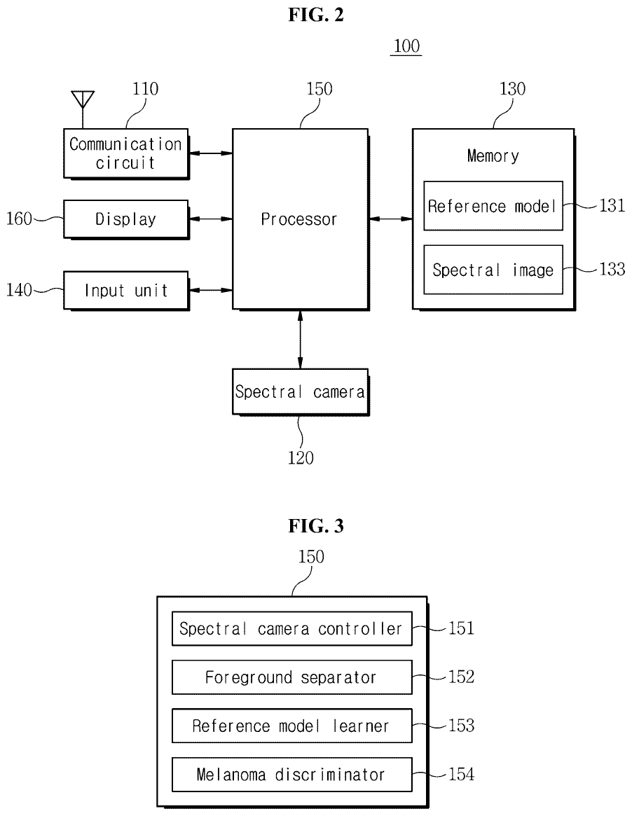

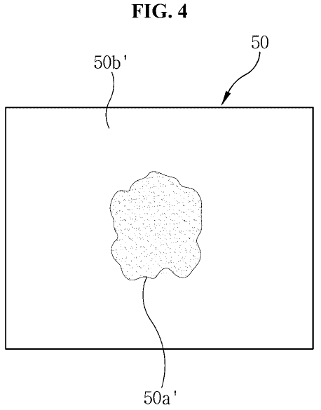

FIG. 2 is a diagram showing an example of components of a melanoma discrimination device according to the first embodiment of the present invention, and FIG. 3 is a diagram showing an example of components of the processor in FIG. 2.

First, referring to FIG. 2, the melanoma discrimination device 100 according to the first embodiment of the present invention may include a communication circuit 110, a spectral camera 120, a memory 130, an input unit 140, a display 160, and a processor 150. In addition, the melanoma discrimination device 100 may further include a mounting structure 129 for mounting the spectral camera 120 such that the at least one spectral camera 120 can capture a spectral image of an examination target 50. In addition, the melanoma discrimination device 100 may further include a power supply (e.g., a permanent power supply or a battery) required for the operation of at least one of the above-mentioned components, for example, the communication circuit 110, the spectral camera 120, the memory 130, the input unit 140, the display 160, and the processor 150.

The communication circuit 110 can support the communication function of the melanoma discrimination device 100. For example, if the server device 200 is designed to perform the calculation required for the melanoma discrimination function according to the first embodiment of the present invention, the communication circuit 110 may transmit at least one spectral image collected by the spectral camera 120 to the server device 200. Meanwhile, the melanoma discrimination function may be performed independently by the melanoma discrimination device 100. In this case, the communication circuit 110 may transmit a message including an examination result according to the melanoma discrimination function to a terminal of an administrator of the melanoma discrimination device 100 or a designated user terminal device under the control of the processor 150. Alternatively, the communication circuit 110 may output (or transmit) the message to the server device 200 under the control of the processor 150. If the melanoma discrimination device 100 includes a separate output device (e.g., the display 160 or an audio device), the message may be output through the output device. The communication circuit 110 may establish a communication channel with an external server device and receive the reference model 131 from the external server device. The reference model 131 may include a model generated through learning on data in which the foreground and background of various melanoma-related spectral images are separated. In this regard, the communication circuit 110 may establish a communication channel with an external server device at regular intervals under the control of the processor 150, and if there is a newly updated reference model 131, may receive the updated reference model 131 from the external server device and store (or update) it in the memory 130.

The spectral camera 120 may be disposed to capture a spectral image of the examination target 50. At least one spectral camera 120 may be disposed, and when a plurality of spectral cameras are disposed, the spectral cameras may be arranged to capture the examination target 50 by dividing it into regions or capture the examination target 50 from various angles. The spectral camera 120 may be activated in response to the control of the processor 150, and when acquiring a spectral image of the examination target 50, it may transmit the acquired image to the processor 150. Alternatively, in response to the control of the processor 150, the spectral image acquired by the spectral camera 120 may be transmitted to the server device 200 via the communication circuit 110.

The memory 130 can store at least one program or data required for the operation of the melanoma discrimination device 100. For example, the memory 130 may temporarily or semi-permanently store a control program required for operating the at least one spectral camera 120 and spectral images 133 acquired through the at least one spectral camera 120. For example, the memory 130 may store the reference model 131 used for comparative analysis with the currently captured spectral image for the examination target 50. The reference model 131 may be received from an external server device as described above. Alternatively, if the plurality of spectral images 133 are accumulatively stored in a predefined amount or more, the reference model 131 may be generated through the operation of the processor 150. In this regard, the memory 130 may store diagnosis results matched with the spectral images 133 (e.g., whether the spectral image 133 is melanoma, information on the type of melanoma, information on at least some of the patient's age, weight, and place of residence, or the like). For example, the reference model 131 may be generated through unsupervised learning on separated data in which the foreground and background of the plurality of spectral images 133 are separated.

The input unit 140 may include various input tools for operating the melanoma discrimination device 100. For example, the input unit 140 may generate, in response to a user's manipulation, at least one of an input signal for activating the spectral camera 120, an input signal for operating the spectral camera 120 to acquire the spectral image 133, an input signal for entering the diagnosis result of the acquired spectral image 133, and an input signal for requesting the output of the analysis result of the spectral image 133. The input unit 140 may include at least one of various tools such as a soft key (or an input tool based on a touch screen or touch pad), a physical key, a voice input device, a gesture input device, and a jog shuttle.

The display 160 can output at least one screen required for operating the melanoma discrimination device 100. For example, the display 160 may output at least one of a screen indicating whether at least one device (e.g., at least one of the spectral camera 120, the mounting structure 129, the communication circuit 110, and the input unit 140) connected to the melanoma discrimination device 100 is in a normal state, a screen related to the activation of the spectral camera 120, a screen related to the acquisition of the spectral image 133 through the spectral camera 120, and a screen related to the melanoma discrimination result according to analysis of the spectral image 133. In addition, when the melanoma discrimination device 100 is operated in conjunction with the server device 200, the display 160 may output at least one of a screen related to access to the server device 200, a screen related to the request for melanoma discrimination on the spectral image 133, and a screen related to the reception of the melanoma discrimination result of the spectral image 133.

The processor 150 can perform at least one of the transmission and processing of signals required for the operation of the melanoma discrimination device 100 and the storage and output of processing results. For example, the processor 150 may control the spectral camera 120 to acquire the spectral image 133, separate the foreground and background of the acquired spectral image 133, perform a comparative analysis on the separated data with the reference model 131 pre-stored in the memory 130, and output the melanoma discrimination result based on the comparative analysis. In this regard, the processor 150 may include components as shown in FIG. 3.

Referring to FIG. 3, the processor 150 may include at least one of a spectral camera controller 151, a foreground separator 152, a reference model learner 153, and a melanoma discriminator 154.

The spectral camera controller 151 can control the spectral camera 120 or at least a part of the mounting structure 129 so that the spectral camera 120 can capture the examination target 50 with a certain resolution or higher. In this regard, the mounting structure 129 may be configured to perform at least one of a linear movement in at least one direction among forward/backward, left/right, and up/down and a rotational movement at a specific angle with respect to the examination target 50 under the control of the spectral camera controller 151. For example, the spectral camera controller 151 may control the spectral camera 120 to obtain a preview image of the examination target 50 where a lesion has occurred, and detect the resolution of the lesion in the preview image. If the resolution of the lesion is lower than a predefined reference value, the spectral camera controller 151 may adjust the mounting structure 129 to change at least one of the shooting distance and shooting angle between the spectral camera 120 and the examination target 50. For example, the spectral camera controller 151 may first adjust the position of the spectral camera 120 to photograph in a direction perpendicular to the center point of the examination target 50 and then automatically adjust the mounting structure 129 so that the resolution of the examination target 50 is higher than or equal to a predefined reference value. In this process, the spectral camera controller 151 may also adjust at least one of the shooting angle and shooting distance of the spectral camera 120 with respect to the examination target 50 so that the ratio of the size of the lesion and the background size of the lesion on the examination target 50 where the lesion has occurred is higher than a predefined value.

When the spectral camera controller 151 completes preparation for shooting the examination target 50, the spectral camera controller 151 can control the spectral camera 120 to acquire at least one spectral image. For example, when the condition that the radio of the lesion size to the background size in the examination target 50 is within a predefined value is satisfied, the spectral camera controller 151 may control to acquire a spectral image for the examination target 50. Alternatively, when the resolution of the lesion in the examination target 50 is greater than or equal to a predefined value, the spectral camera controller 151 may control to acquire a spectral image for the examination target 50. When the spectral images 133 that meet the above-described conditions are accumulatively stored, the melanoma discrimination device 100 may perform unsupervised learning to generate the reference model 131 based on uniform data, thereby deriving more reliable learning results.

The foreground separator 152 can separate the foreground and background from the spectral images collected by the spectral camera controller 151. By separating the foreground (the region of interest) and background (general skin region other than the region of interest) to perform melanoma discrimination using the spectral images such as M1, the foreground separator 152 can achieve higher accuracy in the melanoma discrimination results. The foreground separator 152 may apply a machine learning (e.g., K-nearest neighbor (KNN)) algorithm as a method of separating the foreground and background in the spectral image. In the spectral image, each spatial coordinate may be vector data which is a spectrum having multiple wavelengths. For example, the wavelengths of a single spectral image spectrum may be expressed as vectors in dimension C, the number of which corresponds to H (height)×W (width). The foreground separator 152 may obtain an adjacency matrix by performing KNN on H×W C-dimensional vectors (spectra) to calculate the indexes of vectors most adjacent to each vector. In the adjacency matrix, a vertex (or node) corresponds to each C-dimensional vector (spectrum), and an edge may correspond to a distance between C-dimensional vectors. The distance between C-dimensional vectors may be measured by at least one deep learning-type index of L2 Norm (e.g., a straight-line distance from the origin of a specific vector to the origin of another vector), L1 Norm (the sum of the absolute values of differences between respective vectors), and cosine similarity (e.g., the similarity of the cosine angle between two vectors, −1 to 1, the closer it is to 1, the greater the similarity).

The reference model learner 153 can accumulatively store data in which the foreground and background are separated by the foreground separator 152 for the acquired spectral image 133. If the foreground and background separation data of the acquired spectral image 133 are accumulated to a predefined amount or more, the reference model learner 153 may perform modeling of unsupervised learning using the foreground and background separation data of the spectral image 133. For example, the reference model learner 153 may learn the spectra, in which the foreground and background are separated, individually or collectively in an unsupervised manner (such as Variational Auto-Encoder). For example, the reference model learner 153 may perform unsupervised learning on data consisting only of spectra related to the foreground and thereby generate a foreground-related reference model. Alternatively, the reference model learner 153 may perform unsupervised learning on all spectra related to the foreground and background and thereby generate a foreground and background-related reference model. That is, the reference model learner 153 may perform modeling on various melanoma-related spectral images and generate reference models corresponding to foreground data and foreground and background integrated data. Meanwhile, if the melanoma discrimination device 100 is designed to receive the reference model 131 from the external server device 200, the reference model generating operation of the reference model learner 153 may be omitted.

When a current spectral image for the patient's examination target 50 is received from the spectral camera controller 151 or the spectral image 133 is stored in the memory 130, the melanoma discriminator 154 may collect the spectral images 133 stored in the memory 130 for melanoma discrimination. Also, the melanoma discriminator 154 may transfer the spectral image 133 to the foreground separator 152 and obtain data from the foreground separator 152 in which foreground and background are separated. The melanoma discriminator 154 may perform clustering by analyzing at least one of the separated foreground spectrum of the current spectral image and the spectrum including both foreground and background and compare the clustering result for the spectrum with the reference model 131. In this process, the melanoma discriminator 154 may perform more accurate melanoma discrimination by comparing and analyzing the foreground spectrum with the foreground reference model while excluding the background spectrum, which may be a noise factor in the foreground analysis, and also reduce the amount of computation in the model comparison. In addition, the melanoma discriminator 154 may perform more accurate melanoma discrimination on the examination target 50 by comparing and analyzing the foreground spectrum with the foreground reference model, comparing and analyzing the spectrum including both foreground and background with the reference model including both foreground and background, and synthesizing the results.

In the above-described operation, the melanoma discriminator 154 may detect a cluster (or a cluster of latent representations) most similar to a cluster (or a cluster of latent representations) corresponding to the foreground (or the foreground and background) of the current spectral image from the reference model 131, and identify a diffusion direction for the detected cluster. By referring to the result of the diffusion direction and the examination result pre-recorded in the memory 130, the melanoma discriminator 154 may output at least one of the melanoma discrimination result and the melanoma type of the current spectral image. Additionally, if the melanoma discriminator 154 does not detect a cluster whose similarity with a cluster corresponding to the foreground (or foreground and background) spectrum of the current spectral image is within a predefined range in the reference model 131, it may detect a cluster having the highest similarity (or a cluster of the reference model having the shortest distance value between clusters) and provide melanoma probability information corresponding to the cluster according to the similarity (or according to the distance value) or suggest a detailed tissue examination.

The above-described melanoma discrimination device 100 of the present invention can perform a non-destructive melanoma examination (e.g., examination without skin incision) by separating the foreground and background of the spectral image acquired by the spectral camera and performing a comparison with the reference model, and can also perform more efficient melanoma discrimination by reducing the time and amount of computation required for melanoma discrimination.

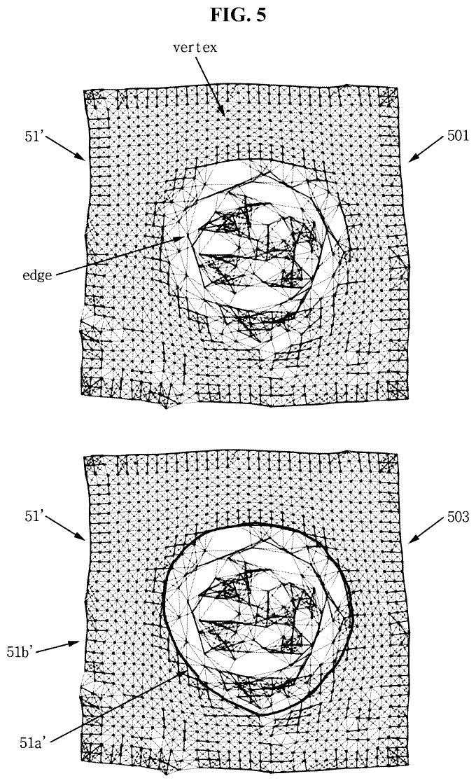

FIG. 4 is a diagram showing an example of a spectral image for an examination target according to the first embodiment of the present invention, and FIG. 5 is a diagram showing an example of separation between foreground and background in the spectral image in FIG. 4.

First, referring to FIG. 4, a spectral image obtained by photographing the examination target 50 with the spectral camera 120 may include spectra of various wavelengths, and when only the wavelengths of the visible light band (e.g., 400 nm to 700 nm) are extracted, an image as illustrated may be displayed. The illustrated image of the examination target 50 may have, for example, a foreground region 50a′ (or a region where a skin lesion has occurred, or a region of interest) and a background region 50b′. The background region 50b′ may be determined according to the definition of the foreground region 50a′.

As described above, the foreground separator 152 of the melanoma discrimination device 100 can convert the spectral wavelengths of the spectral image for the examination target 50 into respective dimensional vectors by using a suitable algorithm (e.g., KNN algorithm) and detect an adjacency matrix 51′ by using the converted dimensional vectors. Screens 501 and 503 of FIG. 5 visualize the adjacency matrix 51′ detected by the foreground separator 152. The adjacency matrix 51′ can be expressed as a plurality of vertices respectively corresponding to C-dimensional vectors (or spectra) and edges corresponding to distance values between respective dimensional vectors. The adjacency matrix 51′ expressed as vertices and edges may include a foreground matrix region 51a′ and a background matrix region 51b′ corresponding to the foreground region 50a′ and the background region 50b′, respectively, described above in FIG. 4.

Meanwhile, as in the 501 screen, if the distance between dimensional vectors (or vertices) in the adjacency matrix 51 expressed by vertices and edges is short, the edge may be displayed short, and if the distance between dimensional vectors is long, the edge may be displayed long. When comparing the central portion of the graph corresponding to the adjacency matrix 51 shown in the 501 screen with the image of the visible light region shown in FIG. 4, it can be seen that a boundary edge between the foreground matrix region 51a′ corresponding to the melanoma area and the background matrix region 51b′ corresponding to the normal skin area is displayed long. Based on this characteristic, the foreground separator 152 can effectively separate the foreground matrix region 51a′ corresponding to the melanoma area from the background matrix region 51b′ by thresholding the edges expressed long beyond a predefined reference value. When separating the foreground matrix region 51a′ through the above-described operation, the foreground separator 152 may define a boundary between the foreground matrix region 51a′ and the background matrix region 51b′, as in screen 503, and efficiently separate the foreground matrix region 51a′ by using the defined boundary.

The reference value (or threshold value) in the above-described thresholding may be specified by the administrator of the melanoma discrimination device 100, and if the administrator fails to or does not specify a separate reference value, the foreground separator 152 may determine a certain reference value (e.g., μ(mean)±1.56σ (standard deviation)) based on the mean and standard deviation of all edges included in the adjacency matrix 51. Here, the constant 1.5 in front of the standard deviation value may be changed according to the user specification. The foreground separator 152 may convert information on the foreground matrix region 51a′ and the background matrix region 51b′ after thresholding into a binary mask form. In this operation, the foreground separator 152 may selectively apply a morphological operation as a method of trimming the edge of the foreground matrix region 51a′.

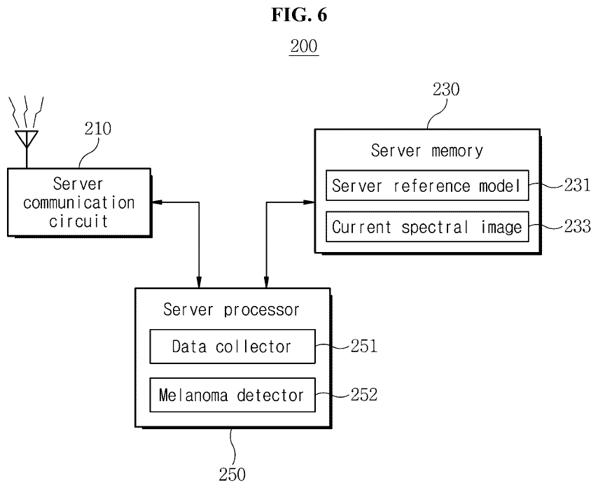

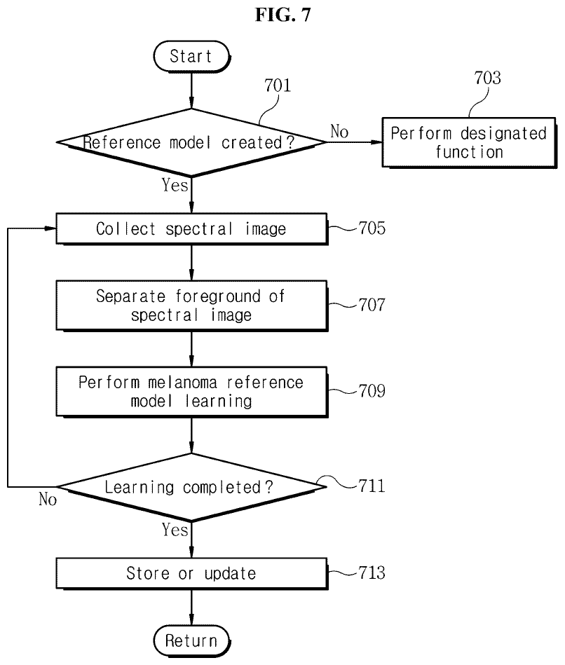

FIG. 6 is a diagram showing an example of components of a server device according to the first embodiment of the present invention. As described above, if the melanoma discrimination device 100 is designed to independently perform the melanoma discrimination function according to the first embodiment of the present invention, the server device 200 may be omitted.

Referring to FIG. 6, the server device 200 may include a server communication circuit 210, a server memory 230, and a server processor 250.

The server communication circuit 210 can establish a communication channel with the melanoma discrimination device 100. The server communication circuit 210 may receive at least one current spectral image from the melanoma discrimination device 100 periodically or in response to occurrence of a predefined event. The server communication circuit 210 may receive a server reference model 231 from an external server device. The server communication circuit 210 may transmit an analysis result for the received at least one current spectral image 233 to the melanoma discrimination device 100 (or a specified user terminal) under the control of the server processor 250.

The server memory 230 can store at least one program or data required for the operation of the server device 200. For example, the server memory 230 may store at least one of the current spectral image 233 collected and transmitted by the melanoma discrimination device 100, and the server reference model 231 for comparison with the current spectral image 233. The current spectral image 233 may be an image corresponding to the above-described spectral image 133 stored in the memory 130 of the melanoma discrimination device 100. For example, the current spectral image 233 may include a spectral image currently acquired for the examination target 50. The server reference model 231 may correspond to the above-described reference model 131 stored in the memory 130 of the melanoma discrimination device 100. For example, the server reference model 231 may be generated by the melanoma discrimination device 100 and provided to the server device 200. Alternatively, the server reference model 231 may be generated by the server processor 250 based on a predetermined amount or more of current spectral images 233 stored in the server memory 230.

The server processor 250 can control the transmission and processing of signals required for the operation of the server device 200, storage or transmission of results, or transmission of messages corresponding to results. In this regard, the server processor 250 may include a data collector 251 and a melanoma detector 252.

The data collector 251 can establish a communication channel with the melanoma discrimination device 100 and receive the current spectral image 233 of the examination target 50 from the melanoma discrimination device 100. Here, the server device 200 may be in a state where the server reference model 231 has been pre-stored, and if there is no server reference model 231, the data collector 251 may receive the server reference model 231 from an external server device. As mentioned above, the server reference model 231 may also be generated through model learning in the server device 200. In this regard, when the server device 200 is operated in a mode of learning the server reference model 231, the data collector 251 may collect various spectral images necessary for generating the server reference model 231 from an external server device or the melanoma discrimination device 100.

The data collector 251 may receive the current spectral image 233 from the melanoma discrimination device 100 in relation to a request for melanoma discrimination for the patient's examination target 50. In this case, the data collector 251 may store the current spectral image 233 together with the identifier information of the melanoma discrimination device 100 in the memory 130 and request the melanoma detector 252 to analyze the current spectral image 233.

When the melanoma detector 252 is notified of receiving the current spectral image 233 from the data collector 251, it can perform an analysis on the current spectral image 233 stored in the server memory 230. For example, the melanoma detector 252 may separate the foreground region and background region of the current spectral image 233, compare the separated foreground and background spectra with the server reference model 231, and thereby perform a melanoma discrimination on the foreground spectrum (or the foreground and background spectra) of the examination target 50. In this operation, the melanoma detector 252 may detect, for example, the directionality of the latent vector for the foreground region and collect the result of whether there is melanoma corresponding to the latent vector directionality for the detected foreground region and the type of melanoma.

The melanoma detector 252 may provide the melanoma discrimination device 100 with a screen indicating the latent vector directionality for at least a portion of the current spectral image 233 (e.g., a foreground region or a region including both foreground and background). Alternatively, the melanoma detector 252 may detect a cluster most similar to a cluster corresponding to the foreground spectrum in the server reference model 231 and, based on this, perform melanoma discrimination of the cluster corresponding to the foreground spectrum. If there is no cluster matching a cluster corresponding to at least some spectral of the current spectral image in the server reference model 231, the melanoma detector 252 may output a melanoma probability value based on a distance value to the most similar cluster.

As described above, the server device 200 according to the first embodiment of the present invention can provide a more accurate melanoma discrimination result by comparing the spectrum of a partial wavelength band for the foreground region where a skin lesion has occurred in the spectral image for the examination target 50 with the pre-stored reference model and then performing melanoma discrimination, and if necessary, it can increase the reliability of the result by performing cross-discrimination (e.g., by integrating the melanoma discrimination for the foreground region only and the melanoma discrimination for both the foreground and background regions).

FIG. 7 is a diagram showing an example of a method for operating a melanoma discrimination device in relation to a melanoma discrimination method according to the first embodiment of the present invention.

Referring to FIG. 7, in the method for operating the melanoma discrimination device 100 in relation to the melanoma determination method according to the first embodiment of the present invention, the processor 150 of the melanoma discrimination device 100 may check in step 701 whether an event requesting the creation of a reference model occurs. The event requesting the creation of the reference model may include, for example, an event of entering the request by an administrator of the melanoma discrimination device 100 through the input unit 140 or an event of receiving a request for generating and providing the reference model from the server device 200. Alternatively, the melanoma discrimination device 100 may be designed to collect various spectral images related to melanoma according to predefined scheduling information and generate a corresponding reference model.

If there is no occurrence of an event related to the creation of a reference model, the processor 150 of the melanoma discrimination device 100 may perform a designated function in step 703. For example, if the reference model has been already stored in the memory 130 or if the reference model has been received from an external server device, the processor 150 of the melanoma discrimination device 100 may provide a melanoma discrimination function based on the reference model stored in the memory 130.

If an event related to the creation of a reference model occurs, the processor 150 of the melanoma discrimination device 100 may collect a melanoma-related spectral image in step 705. In this operation, the processor 150 may access an external server device for providing melanoma-related spectral images and collect the spectral images from the external server device. Alternatively, when the spectral camera 120 connected to the melanoma discrimination device 100 photographs the examination target 50 having a lesion, the processor 150 may store and manage the taken spectral image of the examination target 50 as one of the melanoma-related spectral images in the memory 130. Additionally or alternatively, the processor 150 may collect examination results for the acquired spectral images together, perform information matching, and store the matched information in the memory 130.

The processor 150 may perform foreground separation of melanoma-related spectral images. In relation to foreground separation, the processor 150 may apply a KNN algorithm, etc. to the spectral images and configure an adjacency matrix using dimensional vectors and distance values between the dimensional vectors. The processor 150 may define a foreground matrix region using edges having a length greater than or equal to a reference value in the adjacency matrix, define a boundary of the foreground matrix region, and then extract the foreground matrix region. Upon obtaining the foreground matrix region in this manner, the processor 150 may perform unsupervised learning on the obtained foreground matrix in step 709 to generate a reference model based on the foreground matrix. Thereafter, in step 711, the processor 150 may check whether learning is completed. For example, the processor 150 may determine whether learning is completed by checking whether learning has been performed a predetermined number of times or more or whether learning of various melanoma-related spectral images of a predetermined type or more has been performed. If learning is not completed, the processor 150 may return to step 705 to perform reference modeling learning for the foreground matrix region after foreground separation of spectral images for a predefined number of times or a predetermined type or more.

Meanwhile, the processor 150 may further perform reference modeling learning for spectral images including both foreground and background, in addition to modeling for the foreground matrix region. Therefore, the created reference model may include, for example, at least one of a reference model created based on the foreground region and a reference model based on the spectral images including both foreground and background.

When learning is completed, the processor 150 may store the reference model in the memory 130 or update the previously stored reference model in step 711. Alternatively, the processor 150 may provide the reference model to the server device 200 that requested reference model creation. Thereafter, the processor 150 may return to step 701 and re-perform the subsequent operations, or return to step 703 and perform a designated function.

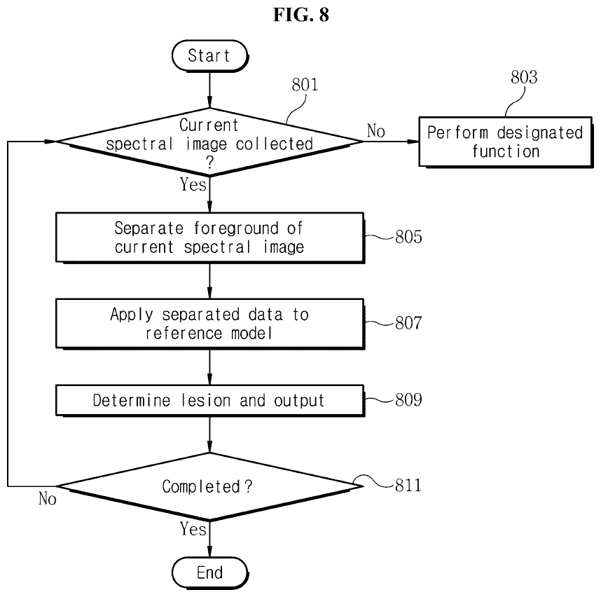

FIG. 8 is a diagram showing another example of a method for operating a melanoma discrimination device in relation to a melanoma discrimination function according to the first embodiment of the present invention.

Referring to FIGS. 1 to 8, in relation to the melanoma discrimination function, the processor 150 of the melanoma discrimination device 100 may check in step 801 whether spectral image collection is requested for melanoma discrimination. In this regard, the melanoma discrimination device 100 may activate a melanoma discrimination application based on spectral images in response to a user input, and activate the spectral camera 120 after the application is activated. If there is no spectral image collection, the processor 150 may perform a designated function in step 803. For example, the processor 150 may enter identification information for the examination target 50 or basic information of the patient (e.g., age, gender, weight, time of lesion occurrence, etc.) in response to a user input.

When spectral image collection is requested, the processor 150 may adjust the distance and angle between the spectral camera 120 and the examination target 50 so that the examination target 50 has a shooting distance or angle to be photographed at a predefined size (or a distance or angle at which the resolution of a certain area including the lesion becomes a predefined resolution). To adjust the predefined distance and angle, the processor 150 may control the spectral camera 120 to acquire a preview image, and through analysis of the acquired preview image, adjust at least one of the distance and angle between the spectral camera 120 and the examination target 50 so that the size of the background area including the lesion becomes the predefined size. When the preparation is completed, the processor 150 may control the spectral camera 120 to acquire a spectral image for the examination target 50. Thereafter, in step 805, the processor 150 may perform a foreground separation of the spectral image. For example, the processor 150 may apply machine learning (e.g., K-nearest neighbor (KNN) algorithm) to the spectral image such as M1 to produce vector data corresponding to a spectrum including multiple wavelengths included in the spectral image. For example, the processor 150 may perform KNN on H (height)×W (width) C-dimensional vectors (spectra) to calculate the indexes of vectors closest to each vector, and thereby obtain an adjacency matrix. In the adjacency matrix, a vertex (or node) corresponds to each C-dimensional vector (spectrum), an edge corresponds to a distance between C-dimensional vectors, and the distance may be measured by indices such as L2, L1, and cosine similarity. The processor 150 may produce a foreground region (or foreground matrix region 51a′) by connecting edges having a length greater than or equal to a predefined reference value.

In step 807, the processor 150 may apply at least some of the separated data to the reference model pre-stored in the memory 130, and in step 809, the processor 150 may discriminate whether the lesion is melanoma, based on the results of being applied to the reference model. Thereafter, the processor 150 may output the melanoma discrimination result to a designated device (e.g., the display 160). In this operation, the processor 150 may obtain the melanoma discrimination result for the foreground region by comparing only the foreground region with the reference model. Alternatively, the processor 150 may perform melanoma discrimination for the foreground region, perform melanoma discrimination by applying both the foreground region and the background region to the reference model, and then output the melanoma determination result through cross-validation. In this operation, the reference model used to perform the melanoma discrimination for the foreground region and the reference model used to perform the melanoma discrimination for the foreground and background regions may be different.

Additionally or alternatively, the processor 150 may output a screen interface on the display 160 through which a medical professional can input the examination results for at least one of information on whether or not there is melanoma and the type of melanoma, and in response to the input of the medical professional, may determine whether or not there is melanoma and the type of melanoma for the examined spectral image. The melanoma discrimination device 100 may maintain the results for the current spectral image as a potential result until the medical professional makes the determination.

In step 811, the processor 150 of the melanoma discrimination device 100 may check whether or not the melanoma discrimination is completed. If an event corresponding to the completion of the melanoma discrimination, such as an input of the completion of the examination by the medical professional or a request to deactivate the spectral camera 120, occurs, the melanoma discrimination may be completed. If no event related to the completion of the melanoma discrimination occurs, the process may return to step 801 and re-perform the subsequent operations.

Meanwhile, although it is described above that the melanoma discrimination device 100 performs operations of acquiring the spectral image for the examination target 50 and determining whether the lesion is melanoma and/or the type of melanoma, the present invention is not limited thereto. Alternatively, the operations described in FIG. 8 may be performed by the server device 200. In this case, the server device 200 may establish a communication channel with the melanoma discrimination device 100 before step 801 and then check in step 801 whether the spectral image is received. When the spectral image is received, the server device 200 may perform operations from step 805 for the received spectral image, determine whether the lesion is melanoma and/or the type of melanoma in step 809, and transmit the determined result back to the melanoma discrimination device 100. In this process, the server device 200 may store and manage the identification information of the melanoma discrimination device 100 providing the spectral image together with the received spectral image so that the determined result can be transmitted to the corresponding melanoma discrimination device 100.

As described above, the melanoma discrimination function based on spectral images according to the first embodiment of the present invention can determine whether or not melanoma is present based on a spectral image of skin suspected of melanoma, and in the melanoma discrimination process, it can increase the reliability of the melanoma discrimination result by separating the foreground and background of the spectral image and utilizing the separated data integrally or partially.

### Second Embodiment

Hereinafter, in the present invention, a melanoma examination function, a melanoma examination system environment supporting the same, and the types and roles of each component included therein will be described.

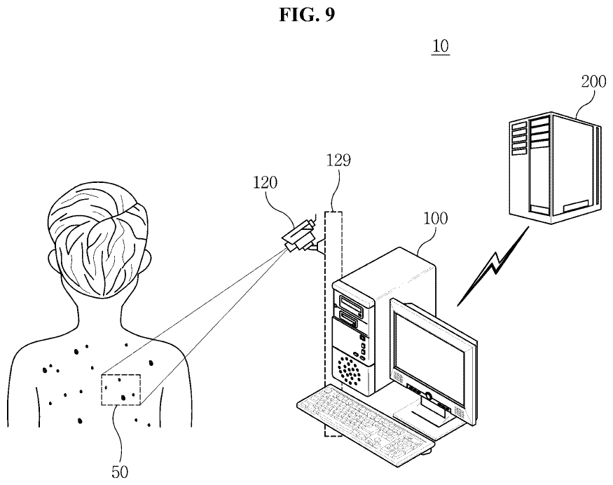

FIG. 9 is a diagram showing an example of a system environment supporting a melanoma examination function according to the second embodiment of the present invention.

Referring to FIG. 9, the system environment 10 that supports the melanoma examination function of the present invention may include an examination target 50 (e.g., at least a portion of the skin of a patient having a lesion (or skin lesion)), a melanoma examination device 100 capable of photographing at least partial area of the examination target 50, and a server device 200 capable of supporting the melanoma examination device 100. In the illustrated system environment 10, a communication channel is formed between the melanoma examination device 100 and the server device 200, and the server device 200 supports the melanoma examination device 100. This is, however, exemplary only and present invention is not limited thereto. In an alternative example, it may be configured to be able to perform spectral image collection and melanoma examination on the examination target 50 based on an embedded program without using both the melanoma examination device 100 and the server device 200. In this case, the server device 200 may be omitted from the configuration of the system environment 10.

The examination target 50 may include the skin of a patient with at least one lesion that may be suspected of being melanoma. Although the patient's back is exemplarily shown as the examination target 50, the present invention is not limited thereto. For example, the examination target 50 may include any area where melanoma may occur. For example, the examination target 50 may be any skin area where melanoma may occur, such as the soles of the feet, palms of the hands, face, chest, abdomen, buttocks, genitals or surrounding areas, scalp, etc.

The melanoma examination device 100 can obtain a spectral image regarding the examination target 50. For example, the melanoma examination device 100 may be configured to approach the examination target 50 within a certain distance and capture a spectral image of the examination target 50 with a resolution of a certain level or higher. In this regard, the melanoma examination device 100 may include at least a spectral camera 120 and a mounting structure 129 that mounts the spectral camera 120. The spectral camera 120 can capture a spectral image regarding the examination target 50. The mounting structure 129 is configured to at least temporarily mount the spectral camera 120 and allow linear movement in at least one of the forward-backward, left-right, and up-down directions of the spectral camera 120 and/or rotational movement at a specific angle. Therefore, the melanoma examination device 100 can adjust the shooting range for the examination target 50 as needed. For example, the melanoma examination device 100 may obtain a spectral image of the examination target 50 including a plurality of lesions by photographing the examination target 50 at a certain distance. Alternatively, the melanoma examination device 100 may obtain a spectral image of the examination target 50 including only one of the plurality of lesions by approaching the examination target 50 within a certain distance and then photographing the examination target 50. The melanoma examination device 100 can perform an analysis on the obtained examination target 50, compare the analysis data with a pre-stored reference model, and based on the results, output information including whether or not it is melanoma and the type of melanoma. Additionally, the melanoma examination device 100 may obtain the results for the examination target 50 through the server device 200. In this case, the melanoma examination device 100 may be configured to transmit the obtained spectral image to the server device 200 without performing a separate analysis and derivation of results on the obtained spectral image, then receive the results from the server device 200, and output the received results. In addition, when the melanoma examination device 100 is configured to independently perform melanoma examination and classification and result output without the server device 200, the melanoma examination device 100 may store in advance the reference model received from an external server device. Alternatively, when the spectral images acquired through the spectral camera 120 and the examination records for the corresponding examination target 50 are accumulatively stored in a predefined amount or more, the melanoma examination device 100 may directly generate a reference model based on unsupervised learning on the stored data. Here, for the purpose of protecting information on personal examination records, the information used to generate the reference model may include only spectral images for the examination target 50 and examination results for the spectral images. Additionally or alternatively, the information used to generate the reference model may include only information other than information that can identify an individual, such as the age, gender, and weight of the examination target 50.

The server device 200 can establish a communication channel with the melanoma examination device 100. The server device 200 can collect at least one spectral image of the examination target 50 from the melanoma examination device 100 and perform analysis on the collected at least one spectral image. In this process, the server device 200 may pre-store a reference model for comparative analysis of the currently acquired spectral image. The reference model may be generated by performing unsupervised learning on the spectral images collected and provided by the melanoma examination device 100, or may be received from a separate external server device that provides the reference model. The server device 200 can provide the result of comparative analysis with the reference model to the melanoma examination device 100. Meanwhile, if the resolution of the acquired spectral image is insufficient or a spectral image with a higher resolution is required, the server device 200 may request the melanoma examination device 100 to adjust the resolution for the examination target 50 with the corresponding lesion, and obtain a spectral image with improved resolution. Meanwhile, if the melanoma examination device 100 is designed to directly output results regarding whether or not the examination target 50 is melanoma and its type, the server device 200 may be omitted from the configuration of the system environment 10.

As described above, the system environment 10 that supports the melanoma examination function according to the second embodiment of the present invention can obtain a spectral image of the examination target 50 and quickly calculate the result of whether the examination target 50 is melanoma through comparison with a pre-learned reference model. In addition, the system environment 10 according to the second embodiment of the present invention can quickly and accurately provide information on the type of melanoma and corresponding treatment plans according to the learning form of the reference model, and can allow a precise tissue examination of the lesion to be performed as needed, thereby supporting saving time and manpower costs for lesion identification.

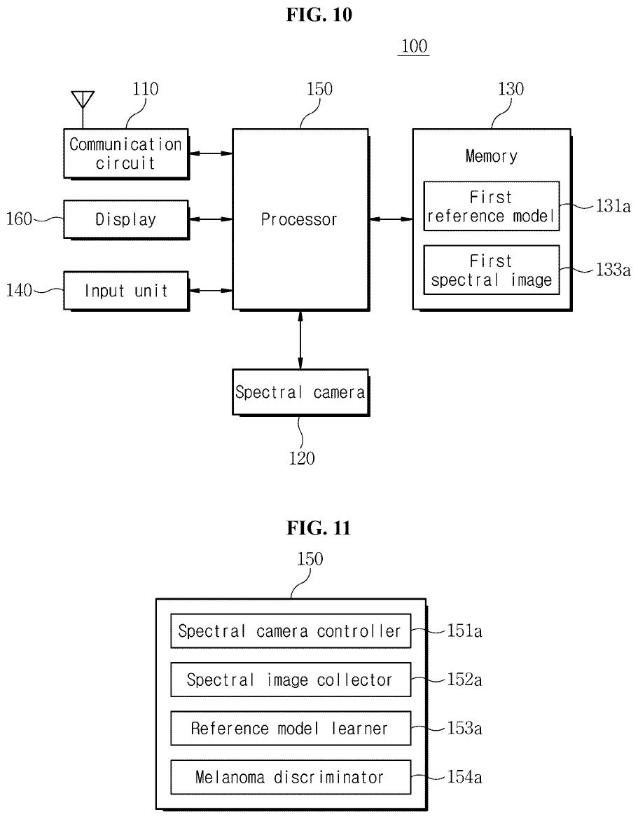

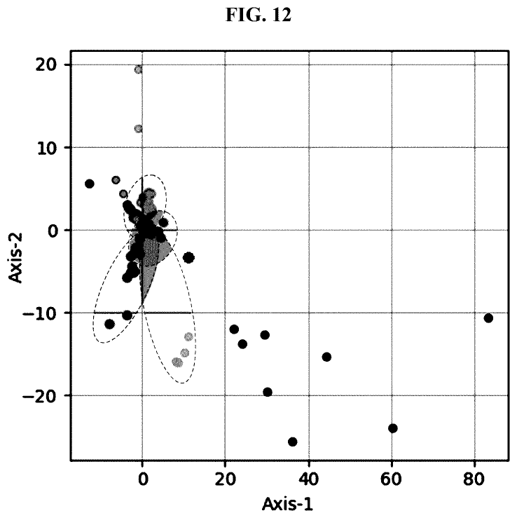

FIG. 10 is a diagram showing an example of components of a melanoma examination device according to the second embodiment of the present invention, and FIG. 11 is a diagram showing an example of components of the processor in FIG. 10. FIG. 12 is a diagram showing an example of a latent vector representation for melanoma data according to the second embodiment of the present invention.

First, referring to FIG. 10, the melanoma examination device 100 according to the second embodiment of the present invention may include a communication circuit 110, a spectral camera 120, a memory 130, an input unit 140, a display 160, and a processor 150. In addition, the melanoma examination device 100 may further include a mounting structure 129 for mounting the spectral camera 120 such that the at least one spectral camera 120 can capture a spectral image of an examination target 50. In addition, the melanoma examination device 100 may further include a power supply (e.g., a permanent power supply or a battery) required for the operation of at least one of the above-mentioned components, for example, the communication circuit 110, the spectral camera 120, the memory 130, the input unit 140, the display 160, and the processor 150.

The communication circuit 110 can establish a communication channel with the server device 200. If the server device 200 is designed to perform the calculation required for the melanoma examination function according to the second embodiment of the present invention, the communication circuit 110 may transmit at least one spectral image collected by the spectral camera 120 to the server device 200. Meanwhile, the melanoma examination function may be performed independently by the melanoma examination device 100. In this case, the communication circuit 110 may transmit a message including an examination result according to the melanoma examination function to a terminal of an administrator of the melanoma examination device 100 or a designated user terminal device under the control of the processor 150. Alternatively, the communication circuit 110 may output (or transmit) the message to the server device 200 under the control of the processor 150. If the melanoma examination device 100 includes a separate output device (e.g., the display 160 or an audio device), the message may be output through the output device.

The communication circuit 110 may establish a communication channel with an external server device and receive a first reference model 131a from the external server device. In this regard, the communication circuit 110 may establish a communication channel with an external server device at regular intervals under the control of the processor 150, and if there is a newly updated first reference model 131a, may receive the updated first reference model 131a from the external server device and store (or update) it in the memory 130.

The spectral camera 120 may be disposed to capture a spectral image of the examination target 50. At least one spectral camera 120 may be disposed, and when a plurality of spectral cameras are disposed, the spectral cameras may be arranged to capture the examination target 50 by dividing it into regions or capture the examination target 50 from various angles. The spectral camera 120 may be activated in response to the control of the processor 150, and when acquiring a spectral image of the examination target 50, it may transmit the acquired image to the processor 150. Alternatively, in response to the control of the processor 150, the spectral image acquired by the spectral camera 120 may be transmitted to the server device 200 via the communication circuit 110.

The memory 130 can store at least one program or data required for the operation of the melanoma examination device 100. For example, the memory 130 may temporarily or semi-permanently store a control program required for operating the at least one spectral camera 120 and the first spectral images 133a acquired through the at least one spectral camera 120. For example, the memory 130 may store a first reference model 131a used for comparative analysis with the spectral image for the examination target 50. The first reference model 131a may be received from an external server device as described above. Alternatively, if the plurality of first spectral images 133a are accumulatively stored in a predefined amount or more, the first reference model 131a may be generated through the operation of the processor 150. In this regard, the memory 130 may store diagnosis results matched with the first spectral images 133a (e.g., whether the first spectral image 133a is melanoma, information on the type of melanoma, information on at least some of the patient's age, weight, and place of residence, or the like). For example, the first reference model 131a may be generated through unsupervised learning on the plurality of first spectral images 133a.

The input unit 140 may include various input tools for operating the melanoma examination device 100. For example, the input unit 140 may generate, in response to a user's manipulation, at least one of an input signal for activating the spectral camera 120, an input signal for acquiring the first spectral image 133a through the spectral camera 120, an input signal for entering the diagnosis result of the acquired first spectral image 133a, and an input signal for requesting the output of the analysis result of the first spectral image 133a. The input unit 140 may include at least one of various tools such as a soft key (or an input tool based on a touch screen or touch pad), a physical key, a voice input device, a gesture input device, and a jog shuttle.

The display 160 can output at least one screen required for operating the melanoma examination device 100. For example, the display 160 may output at least one of a screen indicating whether at least one device (e.g., at least one of the spectral camera 120, the mounting structure 129, the communication circuit 110, and the input unit 140) connected to the melanoma examination device 100 is in a normal state, a screen related to the activation of the spectral camera 120, a screen related to the acquisition of the first spectral image 133a through the spectral camera 120, and a screen related to the melanoma analysis result of the first spectral image 133a. In addition, when the melanoma examination device 100 is operated in conjunction with the server device 200, the display 160 may output at least one of a screen related to access to the server device 200, a screen related to the request for analysis on the first spectral image 133a, and a screen related to the reception of the analysis result of the first spectral image 133a.

The processor 150 can perform at least one of the transmission and processing of signals required for the operation of the melanoma examination device 100 and the storage and output of processing results. For example, the processor 150 may control the spectral camera 120 to acquire the first spectral image 133a, perform a comparative analysis on the acquired first spectral image 133a with the first reference model 131a, and output the melanoma examination result based on the comparative analysis. In this regard, the processor 150 may include components as shown in FIG. 11.

Referring to FIG. 11, the processor 150 may include at least one of a spectral camera controller 151a, a spectral image collector 152a, a reference model learner 153a, and a melanoma discriminator 154a.

The spectral camera controller 151a can control the spectral camera 120 or at least a part of the mounting structure 129 so that the spectral camera 120 can capture the examination target 50 with a certain resolution or higher. In this regard, the mounting structure 129 may be configured to perform at least one of a linear movement in at least one direction among forward/backward, left/right, and up/down and a rotational movement at a specific angle with respect to the examination target 50 under the control of the spectral camera controller 151a. The spectral camera controller 151a may control the spectral camera 120 to obtain a preview image of the examination target 50 where a lesion has occurred, and detect the resolution of the lesion in the preview image. If the resolution of the lesion is lower than a predefined reference value, the spectral camera controller 151a may adjust the mounting structure 129 to change at least one of the distance and shooting angle between the spectral camera 120 and the examination target 50. For example, the spectral camera controller 151a may first adjust the position of the spectral camera 120 to photograph in a direction perpendicular to the center point of the examination target 50 and then automatically adjust the mounting structure 129 so that the resolution of the examination target 50 is higher than or equal to a predefined reference value. In this process, the spectral camera controller 151a may also adjust at least one of the shooting angle and distance of the spectral camera 120 with respect to the examination target 50 so that the ratio of the size of the lesion and the background size of the lesion on the examination target 50 where the lesion has occurred is higher than a predefined value.

When the spectral camera controller 151a completes preparation for shooting the examination target 50, the spectral image collector 152a can control the spectral camera 120 to acquire at least one spectral image. For example, when the condition that the radio of the lesion size to the background size in the examination target 50 is within a predefined value is satisfied, the spectral image collector 152a may control to acquire a spectral image for the examination target 50. Alternatively, when the resolution of the lesion in the examination target 50 is greater than or equal to a predefined value, the spectral image collector 152a may control to acquire a spectral image for the examination target 50. When the first spectral images 133a that meet the above-described conditions are accumulatively stored, the melanoma examination device 100 may perform unsupervised learning to generate the first reference model 131a based on uniform data, thereby deriving more reliable learning results.

The reference model learner 153a can accumulatively store the acquired first spectral images 133a. If the acquired first spectral images 133a are accumulated to a predefined amount or more, the reference model learner 153a may perform modeling of unsupervised learning using the first spectral images 133a. For example, when the first spectral image 133a is acquired, the reference model learner 153a may separate the background and foreground from the acquired first spectral image 133a (roughly performed using K-nearest neighbors (KNN) algorithm, etc.). The reference model learner 153a may perform learning of separated spectrum in an unsupervised manner (such as Variational Auto-Encoder). The reference model learner 153a may input a spectrum corresponding to a test set (e.g., MINST data-set) into the learned model and check the representation of the latent vector. At this time, if the dimension of the latent vector exceeds three dimensions, the reference model learner 153a may reduce, for visualization, the dimension to three or two dimensions by using PCA (principle component analysis, t-SNE (t-distributed stochastic neighbor embedding), UMAP (uniform manifold approximation), etc. As shown in FIG. 12, the reference model learner 153a may express the displayed latent vector in different colors according to predefined lesion information. Here, the expression of different colors may be replaced with, for example, expressions of different shapes, different hatches, or lines of different shapes. In relation to the latent vector information, the center point of latent representation may correspond to the background in the lesion image, and latent representations displayed by spreading based on the center point may indicate each lesion or a normal state. In this case, similar lesions also have similar directions of diffusion of latent representations, and lesions that are close to normal may appear similar to the diffusion direction of normal latent representation. Based on such characteristics, the reference model learner 153a may indicate that the magnitude of a lesion is stronger the farther away the lesion is expressed from the center, and may perform a diagnosis of melanoma or a skin lesion by utilizing the inclusion in the diffusion direction or range of known lesion information. In addition, even if an unknown lesion is input, the reference model learner 153a may provide a possibility of a specific lesion by utilizing the distance (cosine similarity) to the closest clusters among the clusters of existing lesion latent representations. In relation to performing the above-described operation, by referring to the examination results of the first spectral images 133a used for the diffusion directions clustered according to the latent representation results of the latent vector, the reference model learner 153a may enter information on whether or not there is melanoma and its type for the corresponding diffusion directions and generate the first reference model 131a.

The melanoma discriminator 154a may receive a current spectral image for the patient's examination target 50 from the spectral image collector 152a. The melanoma discriminator 154a may perform clustering by analyzing the spectrum of the current spectral image and compare the clustering result for the spectrum with the first reference model 131a. The melanoma discriminator 154a may detect a cluster (or a cluster of latent representations) most similar to a cluster (or a cluster of latent representations) corresponding to the current spectral image from the first reference model 131a, and identify a diffusion direction for the detected cluster. By referring to the result of the diffusion direction and the recorded diagnosis result, the melanoma discriminator 154a may output at least one of whether the current spectral image is melanoma and the type of melanoma. Additionally, if the melanoma discriminator 154a fails to detect a cluster whose similarity is within a predefined range in the first reference model 131a, it may detect a cluster having the highest similarity (or a cluster of the reference model having the shortest distance value between clusters) and provide melanoma probability information corresponding to the cluster based on the similarity (or based on the distance value) or suggest a detailed tissue examination.

The above-described melanoma examination device 100 of the present invention can non-destructively examine melanoma more accurately by using the spectral camera. In addition, the melanoma examination function according to the present invention can reduce the time required for melanoma examination, and can also eliminate the discomfort from the examinee due to skin tissue incision.

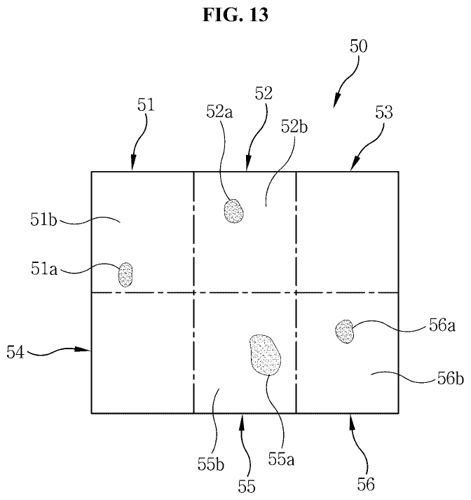

FIG. 13 is a diagram showing an example of a melanoma examination function for an examination target according to the second embodiment of the present invention.

Referring to FIGS. 10 to 13, the processor 150 of the melanoma examination device 100 according to the second embodiment of the present invention can obtain a spectral image corresponding to the examination target 50 as illustrated. Once the spectral image is obtained, the melanoma examination device 100 can perform an analysis on the examination target 50 by using at least some wavelengths of the spectrum of the spectral image. For example, the processor 150 of the melanoma examination device 100 may extract an image by applying some wavelengths (e.g., at least some wavelengths in the visible light wavelength range) of the spectrum of the spectral image for the examination target 50. The processor 150 of the melanoma examination device 100 may divide the extracted image corresponding to the examination target 50 into a plurality of regions including regions where lesions 51a, 52a, 55a, and 56a are disposed. For example, the processor 150 may divide the image extracted from the examination target 50 into six regions 51, 52, 53, 54, 55, and 56. The processor 150 may extract some regions 51, 52, 55, and 56 in which the lesions 51a, 52a, 55a, and 56a are disposed among the six regions 51, 52, 53, 54, 55, and 56, and separate a lesion region and a background region for each of the extracted regions 51, 52, 55, and 56. Referring to the illustrated drawing, the processor 150 may separate a first lesion region 51a and a first background region 51b in the first region 51, separate a second lesion region 52a and a second background region 52b in the second region 52, separate a fifth lesion region 55a and a fifth background region 55b in the fifth region 55, and separate a sixth lesion region 56a and a sixth background region 56b in the sixth region 56. Meanwhile, in the above description, the third lesion region and the third background region and the fourth lesion region and the fourth background region are skipped because the regions are numbered based on the arrangement order. However, the present invention is not limited to such numbering for the lesion regions and background regions. When numbering are for only the lesion regions and background regions, the fifth lesion region and the fifth background region may be referred to as the third lesion region and the third background region, and the sixth lesion region and the sixth background region may be referred to as the fourth lesion region and the fourth background region.

The processor 150 may perform latent vector transformation on each of the lesion regions 51a, 52a, 55a, and 56a and each of the background regions 51b, 52b, 55b, and 56b arranged in the first region 51, the second region 52, the fifth region 55, and the sixth region 56, respectively. The processor 150 may compare the latent vector transformation values for the regions 51, 52, 55, and 56 having lesions with the first reference model 131a described above in FIG. 12 to detect latent vectors having the same or similar directionality. Here, the directionality of the latent vectors included in the first reference model 131a may be matched to the presence or absence of melanoma and the type of melanoma, based on the diagnosis results stored in the memory 130. The processor 150 may output at least some information on whether there is melanoma and the type of melanoma for the regions 51, 52, 55, and 56 having lesions based on information previously stored in the memory 130 and based on a value compared to the first reference model 131a.

As described above, the processor 150 of the melanoma examination device 100 can divide the examination target 50 into a plurality of regions, distinguish regions with lesions and regions without lesions, and perform spectrum analysis only on the regions with lesions among the plurality of regions, thereby reducing the computational burden for the examination target 50 and also providing more accurate information on which lesion is melanoma or what type of melanoma it is through more accurate latent vector comparison of the regions with lesions.

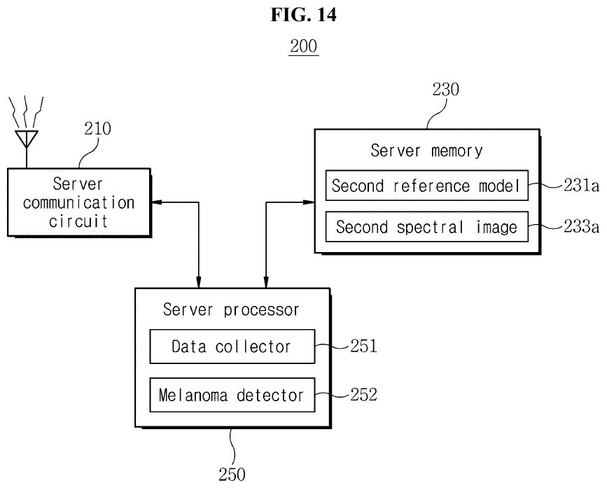

FIG. 14 is a diagram showing an example of components of a server device according to the second embodiment of the present invention. As described above, if the melanoma examination device 100 is designed to independently perform the melanoma examination function according to the second embodiment of the present invention, the server device 200 may be omitted.

Referring to FIG. 14, the server device 200 may include a server communication circuit 210, a server memory 230, and a server processor 250.

The server communication circuit 210 can establish a communication channel with the melanoma examination device 100. The server communication circuit 210 may receive at least one second spectral image from the melanoma examination device 100 periodically or in response to occurrence of a predefined event. The server communication circuit 210 may receive a second reference model 231a from an external server device. The server communication circuit 210 may transmit an analysis result for the received at least one second spectral image to the melanoma examination device 100 (or a specified user terminal) under the control of the server processor 250.

The server memory 230 can store at least one program or data required for the operation of the server device 200. For example, the server memory 230 may store at least one of the second spectral image 233a collected and transmitted by the melanoma examination device 100, and the second reference model 231a for comparison with the second spectral image 233a. The second spectral image 233a may be an image corresponding to the first spectral image 133a described above in the melanoma examination device 100. For example, the second spectral image 233a may include a spectral image currently acquired for the examination target 50. The second reference model 231a may correspond to the above-described first reference model 131a of the melanoma examination device 100. For example, the second reference model 231a may be generated by the melanoma examination device 100 and provided to the server device 200. Alternatively, the second reference model 231a may be generated by the server processor 250 based on a predetermined amount or more of second spectral images 233a stored in the server memory 230.

The server processor 250 can control the transmission and processing of signals required for the operation of the server device 200, storage or transmission of results, or transmission of messages corresponding to results. In this regard, the server processor 250 may include a data collector 251 and a melanoma detector 252.

The data collector 251 can establish a communication channel with the melanoma examination device 100 and receive the second spectral image 233a of the examination target 50 from the melanoma examination device 100. Here, the server device 200 may be in a state where the second reference model 231a has been pre-stored, and if there is no second reference model 231a, the data collector 251 may receive the second reference model 231a from an external server device. As mentioned above, the second reference model 231a may also be generated through model learning in the server device 200. In this regard, when the server device 200 is operated in a mode of learning the second reference model 231a, the data collector 251 may collect various spectral images necessary for generating the second reference model 231a from an external server device or the melanoma examination device 100.

The data collector 251 may receive the second spectral image 233a from the melanoma examination device 100 in relation to a request for melanoma examination for the patient's examination target 50. In this case, the data collector 251 may store the second spectral image 233a together with the identifier information of the melanoma examination device 100 in the memory 130 and request the melanoma detector 252 to analyze the second spectral image 233a.

When the melanoma detector 252 is notified of receiving the second spectral image 233a from the data collector 251, it can perform an analysis on the second spectral image 233a stored in the server memory 230. For example, the melanoma detector 252 may compare the spectrum of the second spectral image 233a with the second reference model 231a, detect the latent vector directionality for the spectral image spectrum of the examination target 50, and collect the result of whether there is melanoma corresponding to the detected latent vector directionality and the type of melanoma. In this process, the melanoma detector 252 may perform an operation similar to the operation of the melanoma discriminator 154a of the processor 150 described above in FIG. 11. For example, when the second spectral image 233a for the inspection target 50 contains a single lesion image, the melanoma detector 252 may perform separation between background and foreground (lesion region) in the acquired image. Alternatively, when the second spectral image 233a contains a plurality of lesion images, the melanoma detector 252 may divide the image into single lesion units as described above in FIG. 13, and perform separation between background and foreground (lesion region) in each region of a single lesion image. The melanoma detector 252 may apply the separated spectrum to the second reference model 231a to identify the representation of the latent vector. In this operation, if the dimension of the latent vector exceeds three dimensions, the melanoma detector 252 may reduce, for visualization, the dimension to three or two dimensions by using PCA, t-SNE, UMAP, etc., and the displayed latent vector may be expressed in different colors according to predefined lesion information.

The melanoma detector 252 may provide the melanoma examination device 100 with a screen indicating the latent vector directionality for the second spectral image 233a. In the screen provided to the melanoma examination device 100, the center point of the latent representation may correspond to the background in the lesion image, and the latent representations displayed by spreading based on the center point may indicate each lesion or a normal state. Here, the melanoma detector 252 may provide diagnosis results (e.g., text corresponding to information on whether or not it is melanoma and the type of melanoma) matching the directionality of each latent representation together with the examination result screen. In addition, the melanoma detector 252 may display a numerical value indicating the magnitude of a lesion according to the distance from the center (e.g., the intensity of the lesion is stronger the farther from the center), and when there is no cluster matching the second reference model 231a, the melanoma probability value may be output based on the distance value with the most similar cluster.

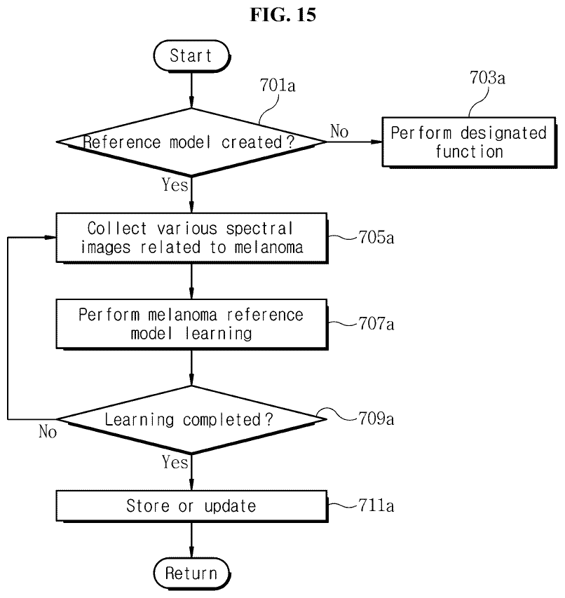

FIG. 15 is a diagram showing an example of a method for operating a melanoma examination device in relation to a melanoma examination method according to the second embodiment of the present invention.

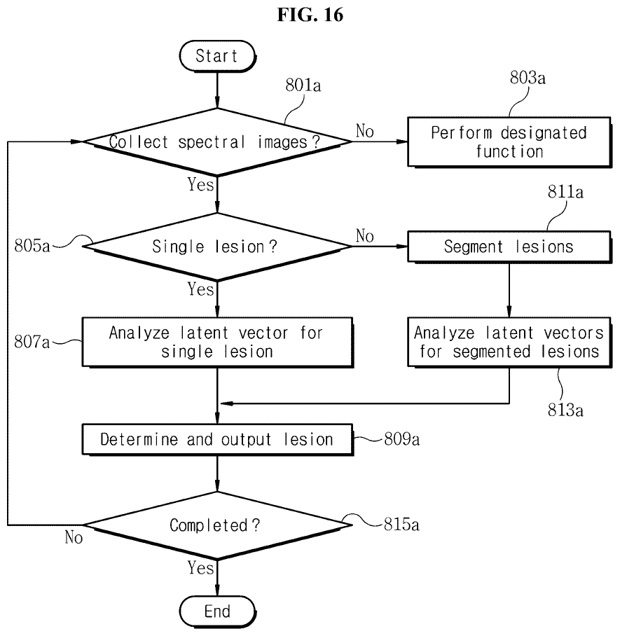

Referring to FIG. 15, in the method for operating the melanoma examination device 100 in relation to the melanoma examination method according to the second embodiment of the present invention, the processor 150 of the melanoma examination device 100 may check in step 701a whether an event requesting the creation of a reference model occurs. The event requesting the creation of the reference model may include, for example, an event of entering the request by an administrator of the melanoma examination device 100 through the input unit 140 or an event of receiving a request for generating and providing the reference model from the server device 200. Alternatively, the melanoma examination device 100 may be designed to collect various spectral images related to melanoma according to predefined scheduling information and generate a corresponding reference model. If there is no occurrence of an event related to the creation of a reference model, the processor 150 of the melanoma examination device 100 may perform a designated function in step 703a. For example, if the reference model has been already stored in the memory 130 or if the reference model has been received from an external server device, the processor 150 of the melanoma examination device 100 may provide a melanoma examination function based on the reference model stored in the memory 130. The operations related to the above melanoma examination function will be described below based on FIG. 16.

If an event related to the creation of a reference model occurs, the processor 150 of the melanoma examination device 100 may collect various melanoma-related spectral images in step 705a. In this operation, the processor 150 may access an external server device for providing various melanoma-related spectral images and collect the spectral images from the external server device. Alternatively, when the spectral camera 120 connected to the melanoma examination device 100 photographs the examination target 50 having a lesion, the processor 150 may store and manage the taken spectral image of the examination target 50 as one of the melanoma-related spectral images in the memory 130. Additionally or alternatively, the processor 150 may collect diagnosis results for the acquired spectral images together, perform information matching, and store the matched information in the memory 130.

The processor 150 may collect a predetermined amount or more of various spectral images related to melanoma. Alternatively, the processor 150 may perform melanoma reference model learning in step 707a each time it collects melanoma-related spectral images. The melanoma reference model learning may be performed by, for example, separating a lesion region and a background region for each of the various spectral images and performing unsupervised learning (e.g., Variational Auto-Encoder) on the spectrum of each separated region to perform modeling. The processor 150 may check whether learning is completed in step 709a. If learning is not completed, the processor 150 may return to step 705a and re-perform the subsequent operations. Alternatively, upon acquiring a new spectral image for learning, the processor 150 may re-perform steps 705a to 707a. When learning is completed, the processor 150 may store the reference model in the memory 130 or update the previously stored reference model in step 711a. Alternatively, the processor 150 may provide the reference model to the server device 200 that requested reference model creation. Thereafter, the processor 150 may return to step 701a and re-perform the subsequent operations, or return to step 703a and perform a designated function.

FIG. 16 is a diagram showing another example of a method for operating a melanoma examination device in relation to a melanoma examination function according to the second embodiment of the present invention.

Referring to FIGS. 9 to 16, in relation to the melanoma examination function, the processor 150 of the melanoma examination device 100 may check in step 801a whether spectral image collection is requested for melanoma examination. In this regard, the melanoma examination device 100 may activate a melanoma examination application based on spectral images in response to a user input, and activate the spectral camera 120 after the application is activated. If there is no spectral image collection, the processor 150 may perform a designated function in step 803a. For example, the processor 150 may enter identification information for the examination target 50 or basic information of the patient (e.g., age, gender, weight, time of lesion occurrence, etc.) in response to a user input.

When spectral image collection is requested, the processor 150 may adjust the distance and angle between the spectral camera 120 and the examination target 50 so that the examination target 50 has a shooting distance or angle to be photographed at a predefined size (or a distance or angle at which the resolution of a certain area including the lesion becomes a predefined resolution). To adjust the predefined distance and angle, the processor 150 may control the spectral camera 120 to acquire a preview image, and through analysis of the acquired preview image, adjust at least one of the distance and angle between the spectral camera 120 and the examination target 50 so that the size of the background area including the lesion becomes the predefined size. When the preparation is completed, the processor 150 may control the spectral camera 120 to acquire a spectral image for the examination target 50. Thereafter, in step 805a, the processor 150 may analyze the spectral image and determine whether it is a single lesion.

If the spectral image includes a single lesion in step 805a, the processor 150 may perform a latent vector analysis on the single lesion in step 807a. In this regard, the processor 150 may apply the acquired current spectral image to a reference model pre-stored in the memory 130 to detect a latent representation.

In step 809a, the processor 150 may analyze the detected latent representation and output a determination result on whether the lesion included in the current spectral image is melanoma and/or the type of melanoma.

On the other hand, if the spectral image includes a plurality of lesions in step 805a, the processor 150 may process lesion segmentation in step 811a. In this process, the processor 150 may divide the spectral image into regions so that the plurality of lesions distributed in the spectral image have a predefined size. In this process, the processor 150 may divide the spectral image in a grid shape or based on mosaics including lesion regions and a background region of a constant size including the lesion.

When the lesion segmentation is completed, the processor 150 may perform latent vector analysis on the segmented lesions in step 813a. In this process, the processor 150 may call the reference model pre-stored in the memory 130 and sequentially apply data on the plurality of lesions to the reference model. Alternatively, the processor 150 may extract latent vectors by applying each data to the reference model in the order of relatively small lesions to relatively large lesions among the plurality of lesions. Thereafter, the processor 150 may return to step 809a to determine whether each of the plurality of lesions is melanoma and the melanoma type, and output the determined result. In this regard, the processor 150 may output the determined result to the display 160 connected to the melanoma examination device 100, or output it to a predefined display device or user terminal.

Additionally or alternatively, the processor 150 may output a screen interface on the display 160 through which a medical professional can input the examination results for at least one of information on whether or not there is melanoma and the type of melanoma, and in response to the input of the medical professional, may determine whether or not there is melanoma and the type of melanoma for the examined spectral image. The melanoma examination device 100 may maintain the results for the current spectral image as a potential result until the medical professional makes the determination.

In step 815a, the processor 150 of the melanoma examination device 100 may check whether or not the melanoma examination is completed. If an event corresponding to the completion of the melanoma examination, such as an input of the completion of the examination by the medical professional or a request to deactivate the spectral camera 120, occurs, the melanoma examination may be completed. If no event related to the completion of the melanoma examination occurs, the process may return to step 801a and re-perform the subsequent operations.

Meanwhile, although it is described above that the melanoma examination device 100 performs operations of acquiring the spectral image for the examination target 50 and determining whether the lesion is melanoma and/or the type of melanoma, the present invention is not limited thereto. Alternatively, the operations described in FIG. 16 may be performed by the server device 200. In this case, the server device 200 may establish a communication channel with the melanoma examination device 100 before step 801a and then check in step 801a whether the spectral image is received. When the spectral image is received, the server device 200 may perform operations from step 805a for the received spectral image, determine whether the lesion is melanoma and/or the type of melanoma in step 809a, and transmit the determined result back to the melanoma examination device 100. In this process, the server device 200 may store and manage the identification information of the melanoma examination device 100 providing the spectral image together with the received spectral image so that the determined result can be transmitted to the corresponding melanoma examination device 100.

As described above, the melanoma examination function based on spectral images according to the second embodiment of the present invention can determine whether or not melanoma is present based on a spectral image of skin suspected of melanoma. When performing the examination on a single lesion image, the background and foreground are separated from the acquired image (roughly performed using KNN, etc.), and when performing the examination on multiple lesion images, the image is divided into single lesion units, the single lesion image is separated into background and foreground, and then the separated spectrum (spectral image) can be learned in an unsupervised manner. The melanoma examination function according to the present invention can input the spectrum for the currently acquired spectral image using the reference model for which learning has been completed, check the representation of the latent vector, and provide at least one result of whether or not there is melanoma and the type of melanoma. Based on this operation, the melanoma examination function of the present invention can provide a melanoma detection result for a lesion in a non-destructive manner, thereby reducing patient rejection and providing faster and more accurate examination results.

While the description contains many specific implementation details, these should not be construed as limitations on the scope of the present disclosure or of what may be claimed, but rather as descriptions of features that may be specific to particular embodiments of particular disclosure.

Also, although the description describes that operations are performed in a predetermined order with reference to a drawing, it should not be construed that the operations are required to be performed sequentially or in the predetermined order, which is illustrated to obtain a preferable result, or that all of the illustrated operations are required to be performed. In some cases, multi-tasking and parallel processing may be advantageous. Also, it should not be construed that the division of various system components are required in all types of implementation. It should be understood that the described program components and systems are generally integrated as a single software product or packaged into a multiple-software product.

The description shows the best mode of the present disclosure and provides examples to illustrate the present disclosure and to enable a person skilled in the art to make and use the present disclosure. The present disclosure is not limited by the specific terms used herein. Based on the above-described embodiments, one of ordinary skill in the art can modify, alter, or change the embodiments without departing from the scope of the present disclosure.

Accordingly, the scope of the present disclosure should not be limited by the described embodiments and should be defined by the appended claims.

### REFERENCE NUMERALS

10: System environment
50: Examination target
100: Melanoma discrimination device
110: Communication circuit
120: Spectral camera
130: Memory
140: Input unit
150: Processor
160: Display
200: Server device
210: Server communication circuit
230: Server memory
250: Server processor

What is claimed is:

1. A melanoma discrimination device based on a spectral image, the device comprising:
a spectral camera acquiring a spectral image of an examination target containing at least one skin lesion; and
a processor functionally connected to the spectral camera and configured to:
control the spectral camera to acquire a current spectral image of the examination target,
separate a foreground region where the at least one skin lesion has occurred and a background region other than the foreground region from the current spectral image, and
compare at least a part of the separated foreground and background regions with a pre-stored reference model to output a melanoma discrimination result for the examination target.

2. The device of claim 1, wherein the processor is configured to:
detect an adjacency matrix based on a plurality of dimensional vectors and edges corresponding to distance values between the plurality of dimensional vectors by applying a nearest neighbor technique to the current spectral image, and
extract the foreground region based on edges greater than or equal to a predefined reference value in the adjacency matrix.

3. The device of claim 2, wherein the processor is configured to:
extract a cluster whose similarity with a cluster corresponding to a spectrum of the foreground region is higher than or equal to a predefined reference value, from the reference model, and
output the melanoma discrimination result for the examination target, based on the cluster extracted from the reference model.

4. The device of claim 3, wherein the processor is configured to:
detect directionality of the cluster corresponding to the spectrum of the foreground region,
extract a cluster having a similar directionality within a certain range from the detected directionality, from the reference model, and
output the melanoma discrimination result for the examination target, based on the cluster extracted from the reference model.

5. The device of claim 3, wherein the processor is configured to:
output the adjacency matrix to a display after visualization.

6. The device of claim 1, wherein the processor is configured to:
output the melanoma discrimination result through cross-validation between a melanoma discrimination result based on the foreground region and a melanoma discrimination result based on both the foreground region and the background region.

7. A melanoma discrimination method based on a spectral image, the method comprising:
by a processor of a melanoma discrimination device, controlling a spectral camera to acquire a current spectral image of an examination target containing at least one skin lesion;
by the processor, separating a foreground region where the at least one skin lesion has occurred and a background region other than the foreground region from the current spectral image; and
by the processor, comparing at least a part of the separated foreground and background regions with a pre-stored reference model to output a melanoma discrimination result for the examination target.

8. The method of claim 7, wherein separating the foreground and background regions includes:
detecting an adjacency matrix based on a plurality of dimensional vectors and edges corresponding to distance values between the plurality of dimensional vectors by applying a nearest neighbor technique to the current spectral image; and
extracting the foreground region based on edges greater than or equal to a predefined reference value in the adjacency matrix.

9. The method of claim 8, wherein outputting the melanoma discrimination result includes one of:
extracting a cluster whose similarity with a cluster corresponding to a spectrum of the foreground region is higher than or equal to a predefined reference value, from the reference model, and outputting the melanoma discrimination result for the examination target, based on the cluster extracted from the reference model; or
detecting directionality of the cluster corresponding to the spectrum of the foreground region, extracting a cluster having a similar directionality within a certain range from the detected directionality, from the reference model, and outputting the melanoma discrimination result for the examination target, based on the cluster extracted from the reference model.

10. The method of claim 7, wherein outputting the melanoma discrimination result includes:
outputting the melanoma discrimination result through cross-validation between a melanoma discrimination result based on the foreground region and a melanoma discrimination result based on both the foreground region and the background region.

11. A server device supporting melanoma discrimination based on a spectral image, the device comprising:
a server communication circuit establishing a communication channel with a melanoma discrimination device; and
a server processor functionally connected to the server communication circuit and configured to:
receive a current spectral image of an examination target containing at least one skin lesion from the melanoma discrimination device,
separate a foreground region where the at least one skin lesion has occurred and a background region other than the foreground region from the current spectral image,
perform melanoma discrimination on the examination target by comparing at least a part of the separated foreground and background regions with a pre-stored reference model, and
transmit a melanoma discrimination result to the melanoma discrimination device.

12. The device of claim 11, wherein the server processor is configured to:
perform the melanoma discrimination through cross-validation between a melanoma discrimination result based on the foreground region and a melanoma discrimination result based on both the foreground region and the background region.

13. A melanoma examination device supporting a melanoma examination function based on a spectral image, the device comprising:
a spectral camera acquiring a spectral image of an examination target containing at least one skin lesion; and
a processor functionally connected to the spectral camera and configured to:
control the spectral camera to acquire a current spectral image of the examination target,
detect a latent vector by applying the current spectral image to a pre-stored reference model, and
determine whether the examination target is melanoma based on directionality and form of a latent representation corresponding to the latent vector.

14. The device of claim 13, wherein the processor is configured to:
control the spectral camera so that a shooting angle and distance of the spectral camera with respect to the examination target become a predefined shooting angle and distance.

15. The device of claim 13, wherein the processor is configured to:
determine a type of melanoma for the examination target based on the directionality and form of the latent representation, and output information on the determined type of melanoma.

16. The device of claim 15, wherein the processor is configured to:
determine that a magnitude of the skin lesion in the current spectral image is stronger the farther away the lesion is expressed from a center of the latent representation.

17. The device of claim 13, wherein the processor is configured to:
when the current spectral image contains a plurality of skin lesions, divide the image into regions including the plurality of skin lesions, separate each skin lesion and a background region of a predetermined size surrounding each skin lesion in the divided regions, and perform sequential melanoma examinations on each of the separated skin lesions and background regions.

18. The device of claim 13, wherein the processor is configured to:
extract a cluster for a spectrum of the current spectral image, detect a cluster of latent representation most similar to a cluster corresponding to the current spectral image from the reference model, determine whether the cluster corresponding to the current spectral image is melanoma and a type of melanoma based on the cluster detected from the reference model, and output determined information.

19. A melanoma examination method based on a spectral image, the method comprising:
by a processor of a melanoma examination device, controlling a spectral camera to acquire a current spectral image of an examination target containing a skin lesion;
detecting a latent vector by applying the current spectral image to a pre-stored reference model; and
determining at least one of whether the examination target is melanoma and a type of melanoma, based on directionality and form of a latent representation corresponding to the latent vector.

20. The method of claim 19, further comprising:
outputting information including the determined at least one of whether the examination target is melanoma and the type of melanoma.

21. The method of claim 19, wherein detecting the latent vector includes:
when the current spectral image contains a plurality of skin lesions, dividing the image into regions including the plurality of skin lesions;
separating each skin lesion and a background region of a predetermined size surrounding each skin lesion in the divided regions; and
sequentially detecting the latent vector on each of the separated skin lesions and background regions.

22. The method of claim 19, wherein determining includes:
extracting a cluster for a spectrum of the current spectral image;
detecting a cluster of latent representation most similar to a cluster corresponding to the current spectral image from the reference model; and
determining whether the cluster corresponding to the current spectral image is melanoma and a type of melanoma based on the cluster detected from the reference model.

23. A server device supporting melanoma examination based on a spectral image, the device comprising:
a server communication circuit establishing a communication channel with a melanoma examination device; and
a server processor functionally connected to the server communication circuit and configured to:
receive a current spectral image of an examination target containing a skin lesion from the melanoma examination device,
detect a latent vector by applying the current spectral image to a pre-stored reference model,
determine at least one of whether the examination target is melanoma and a type of melanoma, based on directionality and form of a latent representation corresponding to the latent vector, and
transmit determined information to the melanoma examination device.

24. The device of claim 23, wherein the server processor is configured to:
extract a cluster for a spectrum of the current spectral image,
detect a cluster of latent representation most similar to a cluster corresponding to the current spectral image from the reference model, and
determine whether the cluster corresponding to the current spectral image is melanoma and a type of melanoma based on the cluster detected from the reference model.
